(19) United States
(12) Patent Application Publication
(10) Pub. No.: US 2020/0054660 A1
(43) Pub. Date: Feb. 20, 2020

(54) DNA METHYLATION PROFILING FOR T-CELL IMMUNOTHERAPY

(71) Applicant: St. Jude Children's Research Hospital, Memphis, TN (US)

(72) Inventors: Benjamin Youngblood, Memphis, TN (US); Hossam Abdelsamed, Memphis, TN (US); Hazem Ghoneim, Memphis, TN (US)

(73) Assignee: St. Jude Children's Research Hospital, Memphis, TN (US)

(21) Appl. No.: 16/467,358

(22) PCT Filed: Dec. 7, 2017
(86) PCT No.: PCT/IB2017/057733

§ 371 Date: Jun. 6, 2019

Related U.S. Application Data
(60) Provisional application No. 62/432,202, filed on Dec. 9, 2016.

(51) Int. Cl. A61K 31/7068; C12N 5/0783; C12Q 1/6827; A61K 35/17; A61K 45/06; A61P 31/14

(52) U.S. Cl. CPC A61K 31/7068; C12N 5/0636; C12Q 1/6827; C12N 2501/999; A61K 45/06; A61P 31/14; A61K 35/17

(57) ABSTRACT

Provided herein are methods and compositions for modulating T-cell activity by altering DNA methylation status. Altering the methylation status of CD8+ T cells can prevent T-cell exhaustion and maintain effector functions during sustained antigen exposure. The methods and compositions can be used to treat symptoms of chronic infections and cancer. Further, the methods and compositions relate to predicting T-cell activity by measuring the methylation status of specific memory cell methylation markers and using the markers to identify and separate populations of CD8 T cell having desired T cell activity. The memory cell methylation markers can further be used to identify subjects with chronic infections or cancer that would benefit from personalized therapy, including immune checkpoint blockade therapy.

### STATEMENT OF FEDERALLY SPONSORED RESEARCH OR DEVELOPMENT

This invention was made with government support from the National Institutes of Health grant R01AI114442. The government has certain rights in the invention.

### FIELD OF THE INVENTION

The invention relates to the field of cell biology and immunology. In particular, the invention relates to a method for modulating T-cell activity by altering DNA methylation status. Altering the methylation status of CD8+ T cells can prevent T-cell exhaustion and maintain effector functions during sustained antigen exposure. The methods and compositions can be used to treat symptoms of chronic infections and cancer.

### BACKGROUND OF THE INVENTION

Antigen-driven clonal expansion and differentiation of naive CD8 T cells initially instills the cells with an effector program that facilitates their ability to directly and indirectly kill antigen presenting cells. However, prolonged stimulation of the cells, as occurs during chronic infection and cancer, results in a progressive suppression of the cell's effector function, commonly referred to as “exhaustion”. During acquisition of the exhausted state, T-cell functional impairment occurs hierarchically, with a progressive loss in expression of the effector cytokines interleukin-2 (IL-2), tumor necrosis factor alpha (TNFα), and interferon γ (IFNγ), respectively. Additionally, the cells undergo a decline in their cytolytic ability and proliferative capacity. Gene-expression profiling of functional vs exhausted CD8 T cells revealed that the development of T-cell exhaustion is characterized by altered metabolism, limited proliferation, and sustained upregulated expression of surface inhibitory receptors (IRs) including programmed cell death protein 1 (PD-1), cytotoxic T lymphocyte antigen 4 (CTLA-4), and T cell immunoglobulin mucin receptor 3 (Tim-3).

Following the discovery that inhibitory receptor (IR) expression on exhausted T cells serves as a mechanism to counteract the activating signals of the T cell receptor, it was determined that blocking the interaction between IRs and their ligands (e.g., anti-CTLA-4, anti-PD-1, anti-PD-L1) could transiently rejuvenate the CD8 T-cell effector response. The concept that IRs serve as an immune checkpoint to turn off the T cell effector response was rapidly translated into therapeutic applications that have proven to be a promising therapeutic approach for the treatment of various cancers (Sharma and Allison, 2015a). While Immune Checkpoint Blockade (ICB) therapy has yielded striking clinical responses, its success is unfortunately limited to a minority of patients with cancer (Sharma and Allison, 2015a) and the mechanism(s) underlying ICB therapy non-responsiveness remain a major challenge for the broader application of this therapeutic approach.

Recent efforts to identify antigen-specific CD8 T cells that retain a potential to respond to ICB therapy have established that the cumulative expression of multiple inhibitory receptors progressively restricts the cells' ability to be rejuvenated. Specifically, exhausted CD8 T cells that co-express Tim3 and PD-1 are less responsive to PD-1 blockade therapy, whereas exhausted CD8 T cells that only express PD-1 have a greater potential for PD-1 blockade mediated rejuvenation. It is now clear that the functional heterogeneity and sensitivity to ICB therapy among the pool of exhausted CD8 T cells is demarcated by the combinatorial expression of multiple inhibitory receptors. Commensurate with the progressive upregulation of IRs and reduced responsiveness to ICB therapy, it has become evident that aspects of the T-cell exhaustion gene-expression program can be reinforced, resulting in stable maintenance of exhaustion-associated features even if the antigen levels are reduced or cleared. Stabilization of T-cell exhaustion programs not only limit the efficacy of ICB treatment but also likely restrict the ability of the rejuvenated cells to generate long-lived immunity after antigen clearance.

Given that exhaustion-associated gene expression programs can be maintained in the absence of antigen, limiting the long-term ability of antigen-specific T cells to mount an effective recall response, we sought to better understand the heritable nature of T-cell transcriptional programs and how they impact on ICB therapy. During cellular differentiation, cell-type-specific gene expression programming is achieved by selective recruitment and/or eviction of transcription factors to regions of chromatin that are accessible for binding. Long-term maintenance of transcription factor accessibility to gene regulatory elements is controlled in part by covalent modifications to histones and DNA that affect chromatin structure, resulting in an “epigenetic memory” of gene expression programs in a dividing population of. While a variety of epigenetic modifications are associated with changes in chromatin accessibility, recent evidence supports the notion that DNA methylation is a critical epigenetic mechanism for establishing stable gene silencing programs. Identification of epigenetic markers would be useful in predicting T cell responsiveness in order to alter treatment programs for chronic infections and cancer to ensure that the effector functions of T cells is preserved.

### SUMMARY OF THE INVENTION

Provided herein are methods and compositions for modulating T-cell activity by altering DNA methylation status. Altering the methylation status of CD8+ T cells can prevent T-cell exhaustion and maintain effector functions during sustained antigen exposure. The methods and compositions can be used to treat symptoms of chronic infections and cancer. Further, the methods and compositions relate to predicting T-cell activity by measuring the methylation status of specific memory cell methylation markers and using the markers to identify and separate populations of CD8 T cell having desired T cell activity. The memory cell methylation markers can further be used to identify subjects with chronic infections or cancer that would benefit from personalized therapy, including immune checkpoint blockade therapy.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Compositions and methods are provided herein for predicting and modulating T-cell activity by altering the methylation profile of the genome of a CD8 T cell. Using a genome-wide methylation analysis, de novo DNA-methylation programs were identified that promote terminal differentiation of exhausted T cells and reveal that these programs persist even in cells that exhibit signs of ICB responsiveness after PD-1 blockade therapy. However, CD8 T cells lacking the acquisition of such methylation programs resist functional exhaustion and display a greater expansion potential after ICB with a broader TCR repertoire diversity. Moreover the methylation status of particular genomic loci can distinguish CD8 cells having the poised effector state.

CD8 T cells undergo activation by interaction of the T-cell receptor (TCR) on the CD8 T cell with antigen bound to MHC-I on antigen presenting cells. Once activated the T cell undergoes clonal expansion to increase the number of cells specific for the target antigen. When exposed to infected or dysfunctional somatic cells having the specific antigen for which the TCR is specific, the activated CD8 T cells release cytokines and cytotoxins to eliminate the infected or dysfunctional cell. The release of specific cytokines and cytotoxins by CD8 T cells in response to an antigen is referred to herein as “effector functions” Likewise, the term “effector potential” refers to the ability of CD8 T cells to activate effector functions upon TCR engagement. The term “T cell activity” refers to any of the following: cytokine production (e.g., IFNγ and IL-2) upon TCR engagement; expression of cytotoxic molecules (e.g., granzyme B and perforin) upon TCR engagement; rapid cell division upon TCR engagement; cytolysis of antigen presenting cells; IL-7 and IL-15 mediated homeostatic proliferation; and in vivo trafficking to lymphoid tissues or sites of antigen presentation. Moreover, “T cell activity” can refer to the persistence of immunological memory in the absence of antigen.

The methods and compositions disclosed herein incorporate the association between the methylation status of particular genomic locus with the activity of a CD8 T cell. The term “methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively. By “hypermethylation” or “increased methylation” is meant an increase in methylation of a region of DNA (e.g., a genomic locus as disclosed herein) that is considered statistically significant over levels of a control population. “Hypermethylation” or “increased methylation” may refer to increased levels seen in a subject over time or can refer to the methylation level relative to the methylation status of the same locus in a naïve T cell.

Moreover, the activity of CD8 T cells can be predicted based on measuring the methylation status of one or more than one genomic locus. Accordingly, a “methylation profile” refers to a set of data representing the methylation states or levels of one or more loci within a molecule of DNA from e.g., the genome of an individual or cells or sample from an individual. The profile can indicate the methylation state of every base in an individual, can comprise information regarding a subset of the base pairs (e.g., the methylation state of specific restriction enzyme recognition sequence) in a genome, or can comprise information regarding regional methylation density of each locus. In some embodiments, a methylation profile refers to the methylation states or levels of one or more genomic loci (e.g., biomarkers) described herein. In more specific embodiments, a methylation profile refers to the methylation status of a gene, promoter, transcription factor, 3′ untranslated region (UTR), or regulator of cellular proliferation.

### II. Methods of Modulating T-Cell Activity

Compositions and methods are provided herein for the modulating T-cell activity of CD8 T cells by altering the methylation profile of the genome of a CD8 T cell. Modulating T-cell activity refers to increase or decreasing T-cell activity relative to an appropriate control. Such modulation, modulating, alteration, or altering includes enhancing or repressing cytokine production (e.g., IFNγ and IL-2), enhancing or repressing expression of cytotoxic molecules (e.g., granzyme B and perforin), enhancing or repressing cell division, enhancing or repressing cytolysis of antigen presenting cells, enhancing or repressing IL-7 and IL-15 mediated homeostatic proliferation, enhancing or repressing in vivo trafficking to lymphoid tissues or sites of antigen presentation. Moreover, modulating T-cell activity can refer to the increase or decrease of immunological memory in the absence of antigen. In specific embodiments, the methylation status or methylation level of at least one genomic locus is decreased in order to increase T-cell activity.

The terms “methylation status” or “methylation level” refer to the presence, absence, and/or quantity of methylation at a particular nucleotide, or nucleotides within a portion of DNA. The methylation status of a particular DNA sequence (e.g., a DNA biomarker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value” or “methylation level.” A methylation value or level can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value, represents the methylation status and can thus be used as a quantitative indicator of methylation status. This is of particular use when it is desirable to compare the methylation status of a sequence in a sample to a threshold value. A “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC).

The terms “measuring” and “determining” are used interchangeably throughout, and refer to methods which include obtaining a subject sample and/or detecting the methylation status or level of a biomarker(s) in a sample. In one embodiment, the terms refer to obtaining a subject sample and detecting the methylation status or level of one or more biomarkers in the sample. In another embodiment, the terms “measuring” and “determining” mean detecting the methylation status or level of one or more biomarkers in a subject sample. Measuring can be accomplished by methods known in the art and those further described herein including, but not limited to, quantitative polymerase chain reaction (PCR). The term “measuring” is also used interchangeably throughout with the term “detecting.”

The methylation status of certain genomic loci, or combinations thereof, can be used to modulate or predict the activity of the corresponding CD8 T cell. Specifically, the methylation status of the loci of effector cytokines, transcription factors, or regulators of cellular proliferation can be used to predict or modulate CD8 T-cell activity. For example, the methylation status of genes, promoters, and/or transcription factors of IFNγ, granzyme K (GzmK), granzyme B (GzmB), Prf1, T-bet, Tcf7, Myc, T-bet, eomesodermin (Eomes), Foxp1, CCR7, and/or CD62L can be used for prediction or modulation of T-cell activity, as described elsewhere herein. In specific CpG sites or “CpG islands” in the genome of a CD8 T cell can be modified in order to modulate T cell activity or can be used to predict T cell activity of the corresponding CD8 T cell. The term “CpG islands” refers to a region of genomic DNA which shows higher frequency of 5′-CG-3′ (CpG) dinucleotides than other regions (i.e., control regions) of genomic DNA. CpG sites can also be found in a region with a low frequency of CpG sites such that the sites do not exist in a CpG island. Methylation of DNA at CpG dinucleotides, in particular, the addition of a methyl group to position 5 of the cytosine ring at CpG dinucleotides, is one of the epigenetic modifications in mammalian cells. CpG islands often harbor the promoters of genes and play a pivotal role in the control of gene expression. In normal tissues CpG islands are usually unmethylated, but a subset of islands becomes methylated during the development of a disease or condition.

The methylation status of an individual genomic locus or the methylation profile of a group of loci or entire genome can be altered in order to modulate T cell activity. For example, the methylation status of a genomic locus or a group of genomic loci can be decreased when compared to a proper control in order to increase T cell activity. Specifically, in some embodiments decreasing the methylation status of a genomic locus disclosed herein can increase cytokine production (e.g., IFNγ and IL-2) upon TCR engagement; increase expression of cytotoxic molecules (e.g., granzyme B and perforin) upon TCR engagement; increase rapid cell division upon TCR engagement; increase cytolysis of antigen presenting cells; extend IL-7 and IL-15 mediated homeostatic proliferation; and increase in vivo trafficking to lymphoid tissues or sites of antigen presentation; or extend immunological memory in the absence of antigen when compared to an appropriate control.

The methylation status of an individual genomic locus or the methylation profile of a group of loci or entire genome can be decreased by contacting the CD8 T cell with a demethylation agent or by any other means of one of skill in the art. Demethylation agents are compounds that can reduce or eliminate DNA methylation. For example, demethylation agents include but are not limited to cytidine analogs such as azacitidine and decitabine which bind DNA methyltransferases. Procaine is a DNA-demethylating agent with growth-inhibitory effects in human cancer cells. Any known demethylation agent can be used in the methods and compositions disclosed herein.

In specific embodiments, the expression of a gene responsible for methylation of DNA can be reduced or eliminated in order to decrease the methylation status of an individual genomic locus or the methylation profile of a group of loci or entire genome. For example, the expression of a DNA methyltransferase can be reduced or eliminated by any means known in the art. DNA methyltransferases (DNA MTase) catalyze the transfer of a methyl group to DNA using S-adenosyl methionine as the methyl donor. De novo methyltransferases recognize something in the DNA that allows them to newly methylate cytosines. These are expressed mainly in early embryo development and they set up the pattern of methylation. Maintenance methyltransferases add methylation to DNA when one strand is already methylated. These MTases work throughout the life of the organism to maintain the methylation pattern that had been established by the de novo methyltransferases. Specific DNA methyltransferases include, but are not limited to, DNMT1, TRDMT1, and DNMT3. In particular embodiments, the expression of DNMT1 is reduced or eliminated in order to decrease the methylation status of an individual genomic locus or the methylation profile of a group of loci or entire genome.

The term “DNA methylation inhibitor” or “demethylation agent” encompasses any known or yet unknown compound or agent that reduces, prevents, or removes methylation of DNA. There are several types of DNA methylation inhibitors known including but not limited to: 1) the “DNA methyltransferase inhibitors” or “DNMTi”, encompassing compounds or agents that reduce the enzyme activity of the methyltransferase in any way, 2) “DNA demethylating agents”, that remove methyl groups from the methylated DNA, and 3) “DNA-methylation inhibitors”, that prevent the introduction of methyl groups into the DNA. Inhibitors of DNA methylation have been widely tested for the treatment of cancer and mostly are analogs of the nucleoside deoxycitidine. Several molecular variations of deoxycytidine have been developed, each modified at position 5 of the pyrimidine ring, as reviewed e.g. in “DNA methyltransferase inhibitors—state of the art”, by J. Goffin & E. Eisenhauer (Annals of Oncology 13: 1699-1716, 2002). This distinctive feature is responsible for inhibiting DNMT. Analogs such as ara-C and gemcitabine, which do not possess this change in the pyrimidine ring, do not inhibit methylation. Exemplary oligodeoxynucleotides are those containing 5-azadeoxycytidine (AzadC), e.g. 5-azacytidine (azacitidine), 5-aza-2′-deoxycytidine (decitabine), 1-β-Darabinofuranosyl-5-azacytosine (fazarabine) and dihydro-5-azacytidine (DHAC); those containing 5-fluorodeoxycytidine (FdC); or those with oligodeoxynucleotide duplexes containing 2-H pyrimidinone, such as zebularine. An alternative mechanism for the inhibition of DNMT is the use of antisense oligodeoxynucleotides (ODNs). These are relatively short synthetic nucleic acids designed to hybridize to a specific mRNA sequence. The hybridization can block mRNA translation and cause mRNA degradation. Such antisense ODNs can be directed against DNMT mRNA and have caused a decrease in DNMT mRNA and protein. MG98 for example is an antisense oligodeoxynucleotide directed against the 3′ untranslated region of DNMT1 mRNA. This agent has shown an ability to inhibit DNMT1 expression without effecting DNMT3. Effects may be synergistic in combination with decitabine. Alternatively, one could use non-nucleoside demethylating agents, such as, but not limited to: (−)-epigallocatechin-3-gallate, hydralazine, procaine, and procainamide. In some embodiments, the DNA methylation inhibitor or demethylation agen is selected from the two classes of DNA methylation inhibitors (non-nucleoside and nucleoside demethylating agents) including: 5-azacytidine (azacitidine), 5-aza-2′-deoxycytidine (5-aza-CdR, decitabine), 1-β-Darabinofuranosyl-5-azacytosine (fazarabine), dihydro-5-azacytidine (DHAC), 5-fluorodeoxycytidine (FdC), oligodeoxynucleotide duplexes containing 2-H pyrimidinone, zebularine, antisense oligodeoxynucleotides (ODNs), MG98, (−)-epigallocatechin-3-gallate, hydralazine, procaine, and procainamide.

The T-cell activity of a CD8 T cell can be modulated (e.g., increased) by contacting the CD8 T cell with a methylation inhibitor. Such contacting can be performed in vivo, wherein the cell is in the body of a subject mammal; in vitro, wherein the cell is propagated in culture; or ex vivo, wherein the cell has been taken from a subject mammal and is preserved in culture. For example, a methylation inhibitor can be administered to a subject in order to achieve contact with a CD8 T cell or can be added to a cell culture medium comprising a CD8 T cell. In specific embodiments, contacting a methylation inhibitor with a CD8 T cell will decrease the methylation status of a particular genomic locus or methylation profile which can increase T-cell activity by enhancing cytokine production (e.g., IFNγ and IL-2), enhancing expression of cytotoxic molecules (e.g., granzyme B and perforin), enhancing cell division, enhancing cytolysis of antigen presenting cells, enhancing IL-7 and IL-15 mediated homeostatic proliferation, enhancing in vivo trafficking to lymphoid tissues or sites of antigen presentation or increasing persistence of immunological memory in the absence of antigen. In specific embodiments, a methylation inhibitor is administered along with ICB therapy to a subject having a chronic infection or cancer.

Reduction (i.e., decreasing) of the expression of gene responsible for methylation of DNA (e.g., DNA MTase) can be achieved by any means known in the art. For example, gene expression can be decreased by a mutation. The mutation can be an insertion, a deletion, a substitution or a combination thereof, provided that the mutation leads to a decrease in the expression of a gene responsible for methylation of DNA. In specific embodiments recombinant DNA technology can be used to introduce a mutation into a specific site on the chromosome. Such a mutation may be an insertion, a deletion, a replacement of one nucleotide by another one or a combination thereof, as long as the mutated gene leads to a decrease in the expression of a gene responsible for methylation of DNA. Such a mutation can be made by deletion of a number of base pairs. In one embodiment, the deletion of one single base pair could render a gene encoding a DNA MTase non-functional, thereby decreasing methylation status of the genomic locus, methylation profile, or methylation status of the entire CD8 T-cell genome, since as a result of such a mutation, the other base pairs are no longer in the correct reading frame. In other embodiments, multiple base pairs are removed e.g. about 100 base pairs. In still other embodiments, the length of the entire gene responsible for methylation of DNA is deleted. Mutations introducing a stop-codon in the open reading frame, or mutations causing a frame-shift in the open reading frame could be used to reduce the expression of an allele of a gene responsible for methylation of DNA.

Other techniques for decreasing the expression of a gene responsible for methylation of DNA are well-known in the art. For example, techniques may include modification of the gene by site-directed mutagenesis, restriction enzyme digestion followed by re-ligation, PCR-based mutagenesis techniques, allelic exchange, allelic replacement, RNA interference, or post-translational modification. Standard recombinant DNA techniques such as cloning the gene encoding a DNA MTase, digestion of the gene with a restriction enzyme, followed by endonuclease treatment, re-ligation, and homologous recombination are all known in the art and described in Maniatis/Sambrook (Sambrook, J. et al. Molecular cloning: a laboratory manual. ISBN 0-87969-309-6). Site-directed mutations can be made by means of in vitro site directed mutagenesis using methods well known in the art.

In some embodiments the expression of a gene responsible for methylation of DNA is reduced using interfering nucleic acids or polypeptides. For example, RNA interference or interfering RNAs (“RNAi”) can be used to decrease the expression of a gene responsible for methylation of DNA. “RNAi” refers to a series of related techniques to reduce the expression of genes (see, for example, U.S. Pat. No. 6,506,559, herein incorporated by reference in its entirety). Older techniques referred to by other names are now thought to rely on the same mechanism, but are given different names in the literature. These include “antisense inhibition,” the production of antisense RNA transcripts capable of suppressing the expression of the target protein and “co-suppression” or “sense-suppression,” which refer to the production of sense RNA transcripts capable of suppressing the expression of identical or substantially similar foreign or endogenous genes (U.S. Pat. No. 5,231,020, incorporated herein by reference in its entirety). Such techniques rely on the use of constructs resulting in the accumulation of double stranded RNA with one strand complementary to the target gene to be silenced. The activity of genes responsible for methylation of DNA as disclosed herein can be reduced using RNA interference including microRNAs and siRNAs.

By “reduces” or “reducing” gene expression is intended to mean, the polynucleotide or polypeptide level of the gene responsible for methylation of DNA is statistically lower than the polynucleotide level or polypeptide level of the same target sequence in an appropriate control or the DNA MTase activity of the cell, plant, or plant part is statistically lower than the DNA MTase activity of an appropriate control cell, plant, or plant part. In particular embodiments, reducing the expression of a gene according to the presently disclosed subject matter results in at least a 95% decrease, at least a 90% decrease, at least a 80% decrease, at least a 70% decrease, at least a 60% decrease, at least a 50% decrease, at least a 40% decrease, at least a 30% decrease, at least a 20% decrease, at least a 10% decrease, or at least a 5% decrease of the gene expression when compared to an appropriate control. In other embodiments, reducing the gene expression results in a decrease of about 3%-15%, 10%-25%, 20% to 35%, 30% to 45%, 40%-55%, 50%-65%, 60%-75%, 70%-90%, 70% to 80%, 70%-85%, 80%-95%, 90%-100% in the gene expression when compared to an appropriate control. In specific embodiments the methylation status or methylation profile of a CD8 T cell is reduced by reducing the expression of at least one gene responsible for DNA methylation. Reducing the methylation status or methylation profile of a CD8 T cell, refers to at least a 95% decrease, at least a 90% decrease, at least a 80% decrease, at least a 70% decrease, at least a 60% decrease, at least a 50% decrease, at least a 40% decrease, at least a 30% decrease, at least a 20% decrease, at least a 10% decrease, or at least a 5% decrease of the methylation status or methylation profile of a CD8 T cell or population of T cells when compared to an appropriate control. Methods to assay for the level of the gene expression, methylation status, methylation profile, the expression of a gene responsible for DNA methylation, or the DNA MTase activity are discussed elsewhere herein and known in the art.

The T-cell activity of any T cell can be modulated (e.g., increased) by contacting the cell with a demethylation agent. For example the T-cell activity of any CD8 T cell (i.e., CD8+ T cell) can be increased by reducing the methylation status of a genomic locus or the methylation profile using the methods disclosed herein. Increase in T-cell activity can refer to at least a 95% increase, at least a 90% increase, at least a 80% increase, at least a 70% increase, at least a 60% increase, at least a 50% increase, at least a 40% increase, at least a 30% increase, at least a 20% increase, at least a 10% increase, or at least a 5% increase of the cytokine production (e.g., IFNγ and IL-2), expression of cytotoxic molecules (e.g., granzyme B and perforin), cell division, cytolysis of antigen presenting cells, IL-7 and IL-15 mediated homeostatic proliferation, in vivo trafficking to lymphoid tissues or sites of antigen presentation or increasing persistence of immunological memory in the absence of antigen when compared to an appropriate control, such as a naïve T cell or unmodified T cell.

In particular embodiments, the CD8 T cell is a T cell having a modified T-cell receptor, such as a CAR T cell. As used herein, a “chimeric antigen receptor” or “CAR” refers to an engineered receptor that grafts specificity for an antigen onto an immune effector cell (e.g., a human T cell). A chimeric antigen receptor typically comprises an extracellular ligand-binding domain or moiety and an intracellular domain that comprises one or more stimulatory domains. In some embodiments, the extracellular ligand-binding domain or moiety can be in the form of single-chain variable fragments (scFvs) derived from a monoclonal antibody, which provide specificity for a particular epitope or antigen (e.g., an epitope or antigen preferentially present on the surface of a cancer cell or other disease-causing cell or particle). The extracellular ligand-binding domain can be specific for any antigen or epitope of interest.

T-cell adoptive immunotherapy is a promising approach for cancer treatment. This strategy utilizes isolated human T cells that have been genetically-modified to enhance their specificity for a specific tumor associated antigen. Genetic modification may involve the expression of a chimeric antigen receptor or an exogenous T cell receptor to graft antigen specificity onto the T cell. By contrast to exogenous T cell receptors, chimeric antigen receptors derive their specificity from the variable domains of a monoclonal antibody. Thus, CAR T cells induce tumor immunoreactivity in a major histocompatibility complex non-restricted manner. To date, T cell adoptive immunotherapy has been utilized as a clinical therapy for a number of cancers, including B cell malignancies (e.g., acute lymphoblastic leukemia (ALL), B cell non-Hodgkin lymphoma (NHL), and chronic lymphocytic leukemia), multiple myeloma, neuroblastoma, glioblastoma, advanced gliomas, ovarian cancer, mesothelioma, melanoma, and pancreatic cancer, among others. In some embodiments, CAR T cells having modulated methylation profiles are administered along with ICB therapy.

In specific embodiments, CAR-CD8 T cells may be adoptively transferred into the patient. Adoptive transfer T cell therapy of methylase-deficient CD8 T cells may also be used in combination with immune checkpoint inhibitors such as antibodies to PD-1/PD-L1 and/or CD80/CTLA4 blockade, small molecule checkpoint inhibitors, interleukins, e.g., IL-2 (aldesleukin).

In some embodiments, T-cell activity is increased in a patient having a chronic infection or cancer. In some embodiments, the chronic infection is a chronic viral infection. For example, T-cell activity can be increased using the methods disclosed herein in a subject infected with influenza A virus including subtype H1N1, influenza B virus, influenza C virus, rotavirus A, rotavirus B, rotavirus C, rotavirus D, rotavirus E, SARS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E, or human immunodeficiency virus (HIV). In particular embodiment, the chronic viral infection is HIV, HCV, and/or herpes virus.

As used herein a “proliferative disease” or “cancer” includes, a disease, condition, trait, genotype or phenotype characterized by unregulated cell growth or replication as is known in the art; including leukemias, for example, acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as osteosarcoma, chondrosarcomas, Ewing's sarcoma, fibrosarcomas, giant cell tumors, adamantinomas, and chordomas; brain cancers such as meningiomas, glioblastomas, lower-grade astrocytomas, oligodendrocytomas, pituitary tumors, schwannomas, and metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of disease related gene expression in a cell or tissue, alone or in combination with other therapies.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e., non-metastatic tumor). Tumors are hallmarks of cancer, a neoplastic disease the natural course of which is fatal. Cancer cells exhibit the properties of invasion and metastasis and are highly anaplastic.

In particular embodiments, a methylation inhibitor can be contacted with a CD8 T cell along with an immune modulating agent. As used herein, an “immune modulating agent” is an agent capable of altering the immune response of a subject. In certain embodiments, “immune modulating agents” include adjuvants (substances that enhance the body's immune response to an antigen), vaccines (e.g., cancer vaccines), and those agents capable of altering the function of immune checkpoints, including the CTLA-4, LAG-3, B7-H3, B7-H4, Tim3, BTLA, KIR, A2aR, CD200 and/or PD-1 pathways. Exemplary immune checkpoint modulating agents include anti-CTLA-4 antibody (e.g., ipilimumab), anti-LAG-3 antibody, anti-B7-H3 antibody, anti-B7-H4 antibody, anti-Tim3 antibody, anti-BTLA antibody, anti-KIR antibody, anti-A2aR antibody, anti CD200 antibody, anti-PD-1 antibody, anti-PD-L1 antibody, anti-CD28 antibody, anti-CD80 or -CD86 antibody, anti-B7RP1 antibody, anti-B7-H3 antibody, anti-HVEM antibody, anti-CD137 or -CD137L antibody, anti-OX40 or -OX40L antibody, anti-CD40 or -CD40L antibody, anti-GALS antibody, anti-IL-10 antibody and A2aR drug. For certain such immune pathway gene products, the use of either antagonists or agonists of such gene products is contemplated, as are small molecule modulators of such gene products. In certain embodiments, the “immune modulatory agent” is an anti-PD-1 or anti-PD-L1 antibody.

Thus, increasing or decreasing the methylation status of a specific genomic locus (i.e., epigenetic modulation) can be combined with blockade of specific immune checkpoints such as the PD-1 pathway. These two therapies need not be given concurrently, but could also be given sequentially, beginning with epigenetic modulation and followed by checkpoint blockade. This is because epigenetic modulation induced alterations in gene expression pattern continue after cessation of treatment of tumor cells (Tsai et al. Cancer Cell 2012, 21: 430-446). As used herein, the term “immune checkpoints” means a group of molecules on the cell surface of CD4+ and CD8+ T cells. These molecules fine-tune immune responses by down-modulating or inhibiting an anti-tumor immune response. Immune checkpoint proteins are well known in the art and include, without limitation, PD-L1, as well as CTLA-4, PD-1, VISTA, B7-H2, B7-H3, B7-H4, B7-H6, 2B4, ICOS, HVEM, PD-L2, CD160, gp49B, PIR-B, KIR, TIM-3, LAG-3, HHLA2, butyrophilins, and BTLA (see, for example, WO 2012/177624). As used herein, “immune checkpoint blockade,” “ICB,” or “checkpoint blockade” refers to the administration of an agent that interferes with the production or activity of immune checkpoint proteins.

In certain embodiments, modified CD8 T cells having decreased methylation as disclosed herein may be used in adoptive T cell therapies to enhance immune responses against cancer. For example, this disclosure relates to methods of treating cancer comprising a) collecting immune cells or CD8 T cells from a subject diagnosed with cancer; b) modifying a DNA MTase gene in the isolated immune cells or CD8 T cells such that the DNA MTase gene has decreased expression thereby producing immune cells or CD8 T cells with reduced methyltransferase activity; c) administering or implanting an effective amount of the immune cells or CD8 T cells with decreased methyltransferase activity into the subject diagnosed with cancer. In specific embodiments, the DNA methyltransferase is DNMT1.

In some embodiments the CD8 T cells modified to decrease the expression of DNMT1, also express a chimeric antigen receptor (CAR) specific to a tumor associated antigen or neoantigen. In certain embodiments, the tumor associated antigen is selected from CD5, CD19, CD20, CD30, CD33, CD47, CD52, CD152(CTLA-4), CD274(PD-L1), CD340(ErbB-2), GD2, TPBG, CA-125, CEA, MAGEA1, MAGEA3, MART1, GP100, MUC1, WT1, TAG-72, HPVE6, HPVE7, BING-4, SAP-1, immature laminin receptor, vascular endothelial growth factor (VEGFA) or epidermal growth factor receptor (ErbB-1). In certain embodiments, the tumor associated antigen is selected from CD20, CD20, CD30, CD33, CD52, EpCAM, epithelial cells adhesion molecule, gpA33, glycoprotein A33, Mucins, TAG-72, tumor-associated glycoprotein 72, Folate-binding protein, VEGF, vascular endothelial growth factor, integrin αVβ3, integrin α5β1, FAP, fibroblast activation protein, CEA, carcinoembryonic antigen, tenascin, Ley , Lewis Y antigen, CAIX, carbonic anhydrase IX, epidermal growth factor receptor (EGFR; also known as ERBB1), ERBB2 (also known as HER2), ERBB3, MET (also known as HGFR), insulin-like growth factor 1 receptor (IGF1R), ephrin receptor A3 (EPHA3), tumor necrosis factor (TNF)-related apoptosis-inducing ligand receptor 1 (TRAILR1; also known as TNFRSF10A), TRAILR2 (also known as TNFRSF10B) and receptor activator of nuclear factor-κB ligand (RANKL; also known as TNFSF11) and fragments thereof.

In certain embodiments, the T-cells specific to a tumor antigen can be removed from a tumor sample (TILs) or filtered from blood. Subsequent activation and culturing is performed outside the body (ex vivo) and then they are transfused into the patient. Activation may be accomplished by exposing the T cells to tumor antigens.

III. Methods for Selecting a Subset of CD8 T cells

Methods and compositions are provided herein for selecting a population of CD8 T cells that have a desired activity based on the methylation status of a specific locus or combination of loci or the methylation profile of a genomic region or complete genome of a CD8 T cell. Selection of a subset of CD8 T cells with a desired activity can be performed by measuring the methylation status of a specific locus or combination of loci or the methylation profile of a genomic region or complete genome of a sample of CD8 T cells in order to predict the T cell activity of the population from which the sample was taken.

The methylation status of any individual locus or a group of loci in the genome of a CD8 T cell can be measured by any means known in the art or described herein. For example, methylation can be determined by methylation-specific PCR, whole genome bisulfite sequencing, locus specific bisulfite sequencing, Ingenuity Pathway Analysis (IPA), the HELP assay and other methods using methylation-sensitive restriction endonucleases, ChIP-on-chip assays, restriction landmark genomic scanning, COBRA, Ms-SNuPE, methylated DNA immunoprecipitation (MeDip), pyrosequencing of bisulfite treated DNA, molecular break light assay for DNA adenine methyltransferase activity, methyl sensitive Southern blotting, methyl CpG binding proteins, mass spectrometry, HPLC, and reduced representation bisulfite sequencing. In some embodiments methylation is detected at specific sites of DNA methylation using pyrosequencing after bisulfite treatment and optionally after amplification of the methylation sites. Pyrosequencing technology is a method of sequencing-by-synthesis in real time. In some embodiments, the DNA methylation is detected in a methylation assay utilizing next-generation sequencing. For example, DNA methylation may be detected by massive parallel sequencing with bisulfite conversion, e.g., whole-genome bisulfite sequencing or reduced representation bisulfite sequencing. Optionally, the DNA methylation is detected by microarray, such as a genome-wide microarray.

In specific embodiments, detection of DNA methylation can be performed by first converting the DNA to be analyzed so that the unmethylated cytosine is converted to uracil. In one embodiment, a chemical reagent that selectively modifies either the methylated or non-methylated form of CpG dinucleotide motifs may be used. Suitable chemical reagents include hydrazine and bisulphite ions and the like. For example, isolated DNA can be treated with sodium bisulfite (NaHSO3) which converts unmethylated cytosine to uracil, while methylated cytosines are maintained. Without wishing to be bound by a theory, it is understood that sodium bisulfite reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonated group can be removed under alkaline conditions, resulting in the formation of uracil. The nucleotide conversion results in a change in the sequence of the original DNA. It is general knowledge that the resulting uracil has the base pairing behavior of thymine, which differs from cytosine base pairing behavior. To that end, uracil is recognized as a thymine by DNA polymerase. Therefore after PCR or sequencing, the resultant product contains cytosine only at the position where 5-methylcytosine occurs in the starting template DNA. This makes the discrimination between unmethylated and methylated cytosine possible.

The methylation status of CpG sites in test and controls samples may be compared by calculating the proportion of discordant reads, calculating variance, or calculating information entropy identifying differentially methylated regions, by quantifying methylation difference, or by gene-set analysis (i.e., pathway analysis), preferably by calculating the proportion of discordant reads, calculating variance, or calculating information entropy. Optionally, information entropy is calculated by adapting Shannon entropy. In some embodiments, gene-set analysis is performed by tools such as DAVID, GoSeq or GSEA. In some embodiments, a proportion of discordant reads (PDR) is calculated. Optionally, each region of neighboring CpG sites (e.g., within a sequencing read) is assigned a consistent status or an inconsistent status before calculating the proportion of discordant reads, variance, epipolymorphism or information entropy. There may be multiple inconsistent statuses, each representing a distinct methylation pattern or class of similar methylation patterns.

The CpG site identified for methylation analysis can be in a genomic feature selected from a CpG island, a CpG shore, a CpG shelf, a promoter, an enhancer, an exon, an intron, a gene body, a stem cell associated region, a short interspersed element (SINE), a long interspersed element (LINE), and a long terminal repeat (LTR). In specific embodiments, the CpG site is in a CpG island, a transcription factor, or a promoter within a given genomic locus.

In some embodiments, T-cell activity can be predicted based on the methylation status of a specific genomic locus or combination of genomic loci, referred to herein as a memory cell methylation marker. Accordingly, a positive memory cell methylation marker refers to markers whose methylation status relative to the corresponding methylation status of the same marker of an appropriate control (e.g., naïve T cell) indicates increased T-cell activity compared to a naïve T cell. Likewise, a negative memory cell methylation marker refers to markers whose methylation status relative to the corresponding methylation status of the same marker of an appropriate control (e.g., naïve T cell) indicates equal or decreased T-cell activity compared to a naïve T cell.

The methylation status of an individual marker can be measured at any location within the memory cell methylation marker locus (“marker locus”). Thus, a memory cell methylation marker can refer to a CpG site within a marker locus. As used herein a marker locus includes, but is not limited to, the genomic region beginning 2 kb upstream of the transcription start site and ending 2 kb downstream of the stop codon for each memory cell methylation marker gene. The marker locus can include the region beginning 1 kb upstream of the transcription start site and ending 1 kb downstream of the stop codon, beginning 500 bp upstream of the transcription start site and ending 500 bp downstream of the stop codon, beginning 250 bp upstream of the transcription start site and ending 250 bp downstream of the stop codon, beginning 100 bp upstream of the transcription start site and ending 100 bp downstream of the stop codon, beginning 50 bp upstream of the transcription start site and ending 50 bp downstream of the stop codon, or beginning 10 bp upstream of the transcription start site and ending 10 bp downstream of the stop codon of the memory cell methylation marker gene. In specific embodiments, the methylation status of an individual memory cell methylation marker can be measured at a CpG site within the genomic locus.

In specific embodiments, demethylation of a CpG site at the CCR7 and/or CD62L locus indicates an increased capacity for T-cells to traffick to sites of antigen presentation. In some embodiments, methylation of a CpG site at the T-bet and/or Eomes locus indicates increased T-cell activity. In certain embodiments, demethylation of a CpG site at the Foxpllocus indicates increased T-cell activity. In some embodiments the methylation status of a CpG site in a transcription factor coding sequence at the T-bet, Eomes, and/or Foxp1 locus indicates increased T-cell activity. In some embodiments, demethylation of a CpG site about 500 bp upstream of the transcription start site (TSS) of the IFNγ coding sequence indicates increased T-cell activity. In some embodiments, demethylation of a CpG site about 500 bp upstream of the TSS of the granzyme K (GzmK) coding sequence indicates increased T-cell activity. In some embodiments, demethylation of a CpG site about 10 bp downstream of the TSS of the granzyme B (GzmB) coding sequence indicates increased T-cell activity. In some embodiments, demethylation of a CpG site about 1 kb upstream of the TSS of the perforin 1 (Prf1) coding sequence indicates increased T-cell activity. In particular embodiments, the demethylation of a CpG site in the promoter sequence of the IFNγ, GzmK, GzmB, and/or Prf1 locus indicates increased T-cell activity. In particular embodiments, methylation status of a CpG site at an effector-associated locus can be used to predict T-cell activity. As used herein, an “effector associated locus” includes the coding sequence of any genes encoding proteins that participate in the effector function of CD8 T cells. Examples of effector associated loci include but are not limited to, CD95, CD122, CCR7, CD62L, T-bet, Eomes, Myc, Tcf7, Foxp1, IFNγ, GzmK, GzmB, and/or Prf1. In particular embodiments, CD122 can be a homeostasis-associated locus, CCR7 and CD62L can be referred to as lymphoid homing loci, and Myc, Tcf7, Tbet, and Eomes can be referred to as memory differentiation associated transcription factors.

Populations of T cells having a desired activity can be selected based on the methylation status of an individual locus or a combination of loci of a sample of T cells taken from the population. In some embodiments, T cell populations are selected based on measurement of the methylation status of any marker locus listed herein. In specific embodiments, selected T-cell populations comprise at least 30%, 40%, 50%, 55%, 60%, 65%, 70%, 75%, 80%, 90%, 95%, or more CD8 T cells having at least one positive memory cell methylation marker. Accordingly, CD8 T cell populations selected by the methods disclosed herein comprising at least 30%, 40%, 50%, 55%, 60%, 65%, 70%, 75%, 80%, 90%, 95%, or more CD8 T cells having at least one positive memory cell methylation marker.

In particular embodiments, a memory cell methylation marker would be unmethylated in a normal sample (e.g., normal or control tissue, or normal or control body fluid, stool, blood, serum, amniotic fluid), most importantly in healthy stool, blood, serum, amniotic fluid or other body fluid. In other embodiments, a biomarker would be hypermethylated in a sample from a subject having or at risk of a chronic infection or cancer at a methylation frequency of at least about 10%, at least about 20%, at least about 30%, at least about 40%, at least about 50%, at least about 60%, at least about 70%, at least about 75%, at least about 80%, at least about 85%, at least about 90%, at least about 95%, or about 100%.

In certain embodiments, the present invention provides for a pharmaceutical composition comprising a demethylating agent, as disclosed herein, a CD8 T cell selected by the method disclosed herein, or comprising a population of CD8 T cells comprising at least 30%, 40%, 50%, 55%, 60%, 65%, 70%, 75%, 80%, 90%, 95%, or more CD8 T cells having at least one positive memory cell methylation marker, as disclosed herein. The demethylating agent, CD8 T cell, or T cell population can be suitably formulated and introduced into a subject or the environment of the cell by any means recognized for such delivery. In some embodiments, the pharmaceutical composition comprises a CAR T cell produced from a CD8 T cell selected based on the identification of at least one positive methylation marker disclosed herein.

Such pharmaceutical compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. In some embodiment a synthetic carrier is used wherein the carrier does not exist in nature. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The pharmaceutical compositions can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies with the T cells disclosed herein can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For a compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an T cell or demethylating agent (including, e.g., a protein, polypeptide, or antibody) can include a single treatment or, preferably, can include a series of treatments.

The pharmaceutical compositions can be included in a kit, container, pack, or dispenser together with instructions for administration.

The present invention provides for both prophylactic and therapeutic methods of treating a subject at risk of (or susceptible to) a chronic disease or infection. “Treatment”, or “treating” as used herein, is defined as the application or administration of a therapeutic agent (e.g., a demethylation agent and/or selected T cell) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., a demethylation agent and/or selected T cell). Subjects at risk for the disease can be identified by, for example, one or a combination of diagnostic or prognostic assays as known in the art. Administration of a prophylactic agent can occur prior to the detection of, e.g., cancer in a subject, or the manifestation of symptoms characteristic of the disease or disorder, such that the disease or disorder is prevented or, alternatively, delayed in its progression.

Another aspect of the invention pertains to methods of treating subjects therapeutically, i.e., altering the onset of symptoms of the disease or disorder. These methods can be performed in vitro (e.g., by culturing the cell with the agent(s)) or, alternatively, in vivo (e.g., by administering the agent(s) to a subject). With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment according to that individual's drug response genotype, methylation profile, expression profile, biomarkers, etc. Pharmacogenomics allows a clinician or physician to target prophylactic or therapeutic treatments to patients who will most benefit from the treatment and to avoid treatment of patients who will experience toxic drug-related side effects.

Therapeutic agents can be tested in a selected animal model. For example, an epigenetic agent or immunomodulatory agent as described herein can be used in an animal model to determine the efficacy, toxicity, or side effects of treatment with said agent. Alternatively, an agent (e.g., a therapeutic agent) can be used in an animal model to determine the mechanism of action of such an agent. Accordingly, methods are provided herein for the treatment or prevention of a chronic infection or cancer by administering a demethylation agent, CD8 T cell, or CAR T cell having a desired T cell activity selected based on the methylation status of at least one memory cell methylation marker.

Embodiments:

1. A method for modulating T-cell activity comprising: modulating the methylation profile of the genome of a CD8 T cell.

2. The method of embodiment 1, wherein methylation of the loci of effector cytokines, transcription factors, and regulators of cellular proliferation is altered.

3. The method of embodiment 1, wherein methylation of the loci of effector cytokines, transcription factors, and regulators of cellular proliferation is decreased.

4. The method of any one of embodiments 1-3, wherein said effector cytokines, transcription factors, and regulators of cellular proliferation comprise at least one of: IFNγ, granzyme K, GzmB, and Prf1, T-bet, Tcf7, Myc, T-bet, eomesodermin (Eomes), Foxp1, CCR7, and CD62L.

5. The method of any one of embodiments 2-4, wherein the methylation of at least one CpG site within said locus is decreased.

6. The method of embodiment 5, wherein said at least one CpG site is located within a promoter sequence or transcription factor sequence.

7. The method of embodiment 6, wherein said promoter sequence or transcription factor sequence is operably linked to a nucleic acid sequence encoding an effector cytokine, transcription factor, or regulator of cellular proliferation.

8. The method of embodiment 7, wherein said effector cytokine, transcription factor, or regulator of cellular proliferation comprises at least one of: IFNγ, granzyme K, GzmB, and Prf1, T-bet, Tcf7, and Myc.

9. The method of any one of embodiments 1-8, wherein modulating the methylation profile comprises contacting said T cell with a demethylation agent to produce a modified CD8 T cell.

10. The method of any one of embodiments 1-8, wherein modulating the methylation profile comprises decreasing the activity of at least one DNA methyltransferase to produce a modified CD8 T cell.

11. The method of embodiment 9, wherein said contacting step occurs in vitro.

12. The method of embodiment 9 or 10, wherein said modified CD8 T cell is administered to a subject.

13. The method of any one of embodiments 1-12, wherein said CD8 T cell is a CAR CD8 T cell.

14. The method of embodiment 12, wherein said subject has a chronic infection or cancer.

15. The method of embodiment 14, wherein said chronic infection is a viral or bacterial infection.

16. The method of embodiment 14, wherein said cancer is a lymphoma, a leukemia, non-small cell lung carcinoma (NSCLC), head and neck cancer, skin cancer, melanoma, or squamous cell carcinoma (SCC).

17. The method of any one of embodiments 14-16, further comprising administering and ICB therapy.

18. A method for selecting a subset of CD8 T cells comprising: measuring the methylation profile of at least one CD 8 T cell; and separating a subset of CD8 T cells comprising at least one positive memory cell methylation marker.

19. The method of embodiment 18, wherein said positive memory cell methylation marker comprises an unmethylated memory cell methylation marker.

20. The method of embodiment 18 or 19, wherein said memory cell methylation marker is located at the transcription factor loci for Tcf7, Myc, T-bet, eomesodermin (Eomes), and/or Foxp1.

21. The method of embodiment 18 or 19, wherein said memory cell methylation marker is located in at least one CpG site in the CCR7 and/or CD62L loci.

22. The method of embodiment 18 or 19, wherein said memory cell methylation marker is located within 1kb of the transcription start site of a nucleic acid sequences encoding IFNγ, granzyme K, GzmB, or Prf1.

23. A population of CD8 T cells selected by the method of any one of embodiments 18-22.

24. A population of CD8 T cells comprising at least 60% CD8 T cells having one or more memory cell methylation marker.

25. The population of CD8 T cells of embodiment 24, wherein said memory cell methylation marker comprises an unmethylated memory cell methylation marker.

26. The population of CD8 T cells of embodiment 24 or 25, wherein said memory cell methylation marker is located at the transcription factor loci for Tcf7, Myc, T-bet, eomesodermin (Eomes), and/or Foxp1.

27. The population of CD8 T cells of embodiment 24 or 25, wherein said memory cell methylation marker is located in at least one CpG site in the CCR7 and/or CD62L loci.

28. The population of CD8 T cells of embodiment 24 or 25, wherein said memory cell methylation marker is located within 2kb of the transcription start site of a nucleic acid sequence encoding IFNγ, granzyme K, GzmB, or Prf1.

29. The population of CD8 T cells of any one of embodiments 23-28, wherein the effector potential of said population is greater than the effector potential of a natural population of CD8 T cells from the same origin.

30. A pharmaceutical composition comprising said population of CD8 T cells of any one of embodiments 23-29.

31. A method of treating a chronic infection or cancer in a subject, said method comprising: administering a demethylation agent to a subject having at least one negative memory cell methylation marker.

32. The method of embodiment 31, further comprising measuring the methylation profile of a population of CD8 T cells originating from said subject.

33. The method of embodiment 31, wherein said negative memory cell methylation marker comprises a methylated memory cell methylation marker.

34. The method of embodiment 33, wherein said memory cell methylation marker is located at the transcription factor loci for T-bet, eomesodermin (Eomes), Tcf7, Myc, and/or Foxp1.

35. The method of embodiment 33, wherein said memory cell methylation marker is located in at least one CpG site in the CCR7 and/or CD62L loci.

36. The method of embodiment 33, wherein said memory cell methylation marker is located within 2 kb of the transcription start site of a nucleic acid sequence encoding IFNγ, granzyme K, GzmB, or Prf1.

37. The method of any one of embodiment 33-36, further comprising administering an ICB therapy.

38. A method of treating a chronic infection or cancer in a subject, said method comprising: decreasing the activity of at least one DNA methyltransferase in a subject having at least one negative memory cell methylation marker.

39. The method of embodiment 5, wherein said DNA methyltransferase is Dnmt3a.

40. The method of embodiment 4, further comprising measuring the methylation profile of a population of CD8 T cells originating from said subject.

41. The method of embodiment 40, wherein said negative memory cell methylation marker comprises a methylated memory cell methylation marker.

42. The method of embodiment 40, wherein said memory cell methylation marker is located at the transcription factor loci for T-bet, eomesodermin (Eomes), Tcf7, Myc, and/or Foxp1.

43. The method of embodiment 40, wherein said memory cell methylation marker is located in at least one CpG site in the CCR7 and/or CD62L locus.

44. The method of embodiment 40, wherein said memory cell methylation marker is located within 2kb of the transcription start site of a nucleic acid sequence encoding IFNγ, granzyme K, GzmB, or Prf1.

45. The method of embodiment 40, wherein said chronic infection is a viral or bacterial infection.

46. The method of embodiment 5, wherein said cancer is: a lymphoma, a leukemia, non-small cell lung carcinoma (NSCLC), head and neck cancer, skin cancer, melanoma, or squamous cell carcinoma (SCC).

47. The method of any one of claim 40-46, further comprising administering an ICB therapy.

48. Use of the pharmaceutical composition of embodiment 30 in the treatment of a chronic infection or cancer.

49. The use according to embodiment 48, wherein wherein said chronic infection is a viral or bacterial infection.

50. The method of embodiment 48, wherein said cancer is: a lymphoma, a leukemia, non-small cell lung carcinoma (NSCLC), head and neck cancer, skin cancer, melanoma, or squamous cell carcinoma (SCC).

### EXPERIMENTAL

### Example 1

Treatment of LAP-deficient Mice with PPAR and LXR Agonists to Restore IL-10 Production.

To determine if newly acquired DNA-methylation programs reinforce T-cell exhaustion, a conditional deletion strategy was used to delete de novo DNA methyltransferase 3a (Dnmt3a) in activated CD8 T cells. We report here, using the well-established chronic lymphocytic choriomeningitis virus (LCMV) mouse model of T-cell exhaustion, that de novo DNA methylation acquired during and after the peak of the effector response is critical for establishing T-cell exhaustion. Genome-wide de novo DNA-methylation programs were identified that promote terminal differentiation of exhausted T cells and reveals that these programs persist even in cells that exhibit signs of ICB responsiveness after PD-1 blockade therapy. In contrast, CD8 T cells lacking the acquisition of such methylation programs resist functional exhaustion and display a greater expansion potential after ICB with a broader TCR repertoire diversity. These data establish Dnmt3a mediated de novo DNA methylation as a mechanism restricting the efficacy of ICB therapy and have broad implications for novel approaches to enhance T cell-based immunotherapies.

### Post-effector De Novo DNA-Methylation Programming Promotes T-cell Exhaustion.

Phenotypic and functional changes that occur during the naïve-to-effector stage of CD8 T cell differentiation are accompanied by genome wide changes in DNA-methylation; however, the role of these changes in regulating the functional state of the cell are largely unknown. Furthermore, if exposure to their cognate antigen persists past the effector stage of the immune response, antigen-specific CD8 T cells continue to modify their phenotypic and functional properties yet it is unknown whether this post-effector adaptation is accompanied by additional newly established epigenetic modifications. To elucidate the biological consequence of de novo DNA Methylation programming during the development of T-cell exhaustion, we measured the quantity and function of antigen-specific CD8 T cells in wild-type (WT) mice in which Dnmt3a expression is intact or transgenic mice in which Dnmt3a is conditionally knocked out (Dnmt3a cKO; hereafter referred to as cKO mice) by Cre recombinase under the control of the granzyme b promoter.

To establish an environment where we could monitor changes in the quantity, effector functions, and epigenetic programs of WT and Dnmt3a cKO antigen-specific CD8 T cells during persistent exposure to their cognate antigen, we utilized a well-established model of CD8 T cell exhaustion, in which CD4 T cell-depleted mice are infected with the chronic strain of LCMV (Clone 13). This model establishes a lifelong chronic infection with high viral loads and results in heightened development of T-cell exhaustion. Indeed, LCMV viral loads in the serum of WT and cKO mice remained high for several months. Longitudinal tracking of virus-specific CD8 T cells in the peripheral blood of chronically infected WT mice revealed a progressive decline in gp33-specific (a dominant LCMV epitope) CD8 T cells as well as a reduction in the total quantity of LCMV-specific CD8 T cells (The total pool of virus-specific CD8 T cells was defined as CD44hi PD-1+ CD8 T cells). In contrast, the contraction of virus-specific cKO CD8 T cells after the peak of the effector response was modest, and the quantity of cKO virus-specific CD8 T cells that survived the contraction stage of the immune response was maintained at a much greater level relative to the WT CD8 T cells.

After observing the maintenance of a greater quantity of virus-specific cKO CD8 T cells during chronic infection, we next assessed whether the retained cKO CD8 T cells also maintained their effector function despite persistent antigen exposure. Splenocytes were isolated from chronically infected WT or cKO mice at 2 months post-infection and antigen-specific CD8 T cells were stimulated with the LCMV gp33-41 peptide to measure their capacity to produce effector cytokines IFNγ and IL-2. Antigen specific WT CD8 T cells were severely impaired in their ability to produce IFNγ and IL-2. In contrast, cKO CD8 T cells retained a substantial capacity to co-produce both cytokines. We found that antigen-specific cKO CD8 T cells maintained higher expression of CD44. These data demonstrate that Dnmt3a-deficient CD8 T cells resist the development of functional exhaustion. Notably, both WT and cKO virus-specific CD8 T cells sustained elevated expression of PD-1 over the 2 months of chronic infection, further indicating that these cells were persistently exposed to their cognate antigen and experienced continuous TCR stimulation. However, the cKO cells expressed higher levels of TCR. Taken together, these results demonstrate that Dnmt3a cKO CD8 T cells fail to suppress the expression of their effector cytokines despite prolonged TCR stimulation and sustained PD-1 expression. These data suggest that changes in epigenetic programming are not merely associated with the functional exhaustion of T cells but are in fact necessary to establish a hallmark of T-cell exhaustion.

To identify the de novo DNA-methylation programs associated with the progressive commitment to T-cell exhaustion, we next sought to measure genome-wide DNA-methylation changes in WT and cKO virus-specific CD8 T cells at the effector and exhaustion-stages of the immune response. In order to achieve nucleotide-resolution of genome-wide methylation profiles, whole-genome bisulfite sequencing (WGBS) was performed using genomic DNA from virus-specific CD8 T cells isolated at 8 or 35 days post-infection (dpi). Initial assessment of CpG methylation levels across the entire genome of all samples demonstrated that naïve CD8 T cells have a markedly higher level of genome-wide methylation relative to antigen-specific WT and cKO CD8 T cells isolated at day 8 and 35 post infection.

Using our WGBS data sets, we performed an unsupervised principle-component analysis (PCA) of the methylation status of all CpG sites with >5× coverage in all WT and cKO antigen-specific CD8 T cells to broadly assess the overall relationship between changes in DNA-methylation programming and the differentiation status of the cells. PCA of the WGBS profiles grouped the 35 dpi cKO CD8 T cells with the 8 dpi WT and cKO effector cells, whereas the exhausted WT cells were segregated from the effector cells. These results indicate that de novo DNA-methylation programming acquired after the peak of the effector response is a primary mediator of the progressive decline in WT CD8 T cells effector functions.

To further characterize DNA-methylation changes that delineate effector compared to exhausted CD8 T cells, we parsed the differentially methylated regions (DMRs) into methylation vs demethylation events that arise during the effector-to-exhaustion stage of WT T-cell differentiation. Approximately 1200 DMRs had an increase in the level of DNA methylation (a threshold of 20% change in methylation ratio and p-value <0.01 was used as a cutoff) during the effector-to-exhaustion transition, whereas only ˜280 DMRs were demethylated during this stage of the immune response. These data demonstrate that the majority of DNA-methylation reprogramming during the effector-to-exhaustion stage of the immune response are indeed de novo epigenetic events.

To identify which of these newly methylated programs are mediated by Dnmt3a, we identified the DMRs among WT and cKO CD8 T cells at 35 dpi and compared those regions with the DMRs that gain methylation programs during the effector- to exhausted-state transition in WT cells. We next generated a dendrogram of the WGBS data sets on the basis of the 3000 most variable CpGs among WT and cKO CD8 T cells (FIG. 1G). Quite surprisingly, measurement of Euclidian distances between each population revealed that the replicate data for exhausted WT cells was most closely related to naïve cells. These data demonstrate that post-effector de novo DNA methylation contribute to the development of T-cell exhaustion by re-establishing repressive epigenetic states that were previously present in naïve cells.

We next sought to determine if the Dnmt3a-targeted loci were involved in biological processes known to be directly impacted during T-cell exhaustion. To broadly characterize the cellular functions of exhaustion-specific de novo DNA-methylation programming, we performed ingenuity-pathway analysis (IPA) of all Dnmt3a-targeted genes and identified several potential regulators linked to these de novo epigenetic events. Several transcription factors and signaling molecules that regulate immune-related pathways, including CREBBP (a coactivator of several transcription factors including c-Myc), ID2, ID3, and IFNγ, were identified as putative regulators of the Dnmt3a-targeted loci. Further inspection of the list of exhaustion-associated DMRs targeted by these upstream regulators revealed enrichment of genes that are broadly associated with T-cell effector function, cellular proliferation, and exhaustion-fate commitment. Specifically, the IFNγ, Myc, Tcf7, Ccr7, T-bet, and Eomesodermin (Eomes) loci were among target genes whose expression are intimately coupled to the hallmarks of T-cell exhaustion. Due to the direct association of these genes with the various functional hallmarks of T cell exhaustion, our next series of experiments focused on in-depth characterization of individual DMRs that are representative of each of these T-cell exhaustion hallmarks: repression of effector cytokines, T cell exhaustion functional heterogeneity/fate commitment, and cellular proliferation.

Our data demonstrate that de novo DNA-methylation programs are required to establish T-cell exhaustion and that specific DMRs serve as an epigenetic signature for exhausted T cells. However, it is unclear whether these post-effector DNA-methylation programs are acquired due to a hard-wired differentiation program that continues regardless of additional TCR stimulation or rather due to persistent stimulation of the cells. Therefore, we next sought to examine whether exhaustion-associated de novo DNA-methylation programs were also acquired in highly functional WT memory CD8 T cells generated during an acute viral infection. We designed a loci-specific bisulfite sequencing assay to assess the methylation status of exhaustion-associated DMR in the IFNγ locus in naïve and virus-specific CD8 T cells isolated from chronically infected and infection matched immune (2 months after acute LCMV infection) WT and cKO mice. Ex vivo stimulation of splenocytes from immune or chronically infected WT and cKO mice with the gp33 peptide showed that both memory T cells from immune mice and cKO T cells from chronically infected mice retain high expression of IFNγ. Genomic DNA was then isolated from purified tetramer+CD8 T cells and we performed a loci-specific assay to determine the methylation status of the exhaustion-associated IFNγ DMR in the highly functional memory CD8 T cells. Quite clearly, our results demonstrate that highly functional memory CD8 T cells and cKO cells from chronically infected mice both remain demethylated at the DMR in the IFNγ locus, but only WT cells from chronically infected animals remethylate this region. Thus, acquisition of the post-effector de novo DNA-methylation program at the IFNγ locus during the development of exhaustion is not simply due to slow accumulation of DNA-methylation marks over time in the aged cells but requires chronic stimulation.

Several genes that are normally downregulated at the effector stage of the immune response remain downregulated in exhausted T cells and eventually this program becomes reinforced. Once such gene, Ccr7, is repressed in all effector cells but is then re-expressed in long-lived memory CD8 T cells. To determine if reinforced effector stage downregulation of CCR7 is coupled to persistent stimulation, we measured the methylation level of the DMR in the Ccr7 locus in naïve, effector, and virus-specific WT and cKO CD8 T cells at 60 dpi from acute and chronically infected mice. Indeed, downregulation of CCR7 in effector CD8 T cells accompanied de novo methylation of the locus. Functional memory cells had a reduction in this de novo program consistent with a subset of functional memory CD8 T cells re-expressing CCR7. However, the Ccr7 effector-associated DMR underwent further methylation in the exhausted WT cells. These data suggest that the transcriptional repression of genes during the effector stage of the immune response may become imprinted during or shortly after the effector stage of the immune response.

### De Novo DNA-Methylation Programming Regulates Development of Fully Exhausted T Cells.

Progressive adaptation of antigen-specific cells to chronic stimulation occurs asynchronously among the pool of antigen-specific CD8 T cells and results in heterogeneous populations of CD8 T cells with varying degrees of T-cell exhaustion. Coupled to this adaptation is the progressive co-expression of multiple inhibitory receptors (e.g., PD-1 and Tim-3) and differential expression of the T-box transcription factors T-bet and Eomes. Based on these findings it has been reasoned that the spectrum of cellular plasticity among the pool of antigen-specific CD8 T cells is demarcated by high T-bet and low Eomes expression among partially-exhausted CD8 T cells vs lower T-bet and higher Eomes expression among fully exhausted cells. Our WGBS revealed exhaustion-associated de novo programs in the T-bet and Eomes loci. These results, as well as the preserved effector function of cKO cells, prompted us to measure T-bet and Eomes expression in virus-specific WT and cKO CD8 T cells isolated from chronically infected mice. Dnmt3a-deficient CD8 T cells had significantly higher T-bet and lower Eomes expression compared to those levels in exhausted WT cells. Furthermore, the Eomeslo cells were predominantly Tim-3−, T-bet+, consistent with a previous report that upregulation of Eomes expression during chronic viral infection is coupled to Tim-3 expression. These data suggest that post-effector de novo DNA-methylation programs reinforce the terminal differentiation of exhausted T cells.

Gene-expression profiling of exhausted T cells has revealed a significant downregulation in Tcf7 expression, suggesting Tcf7 has an important role in establishing memory T cells. Consistent with previous reports on the temporal downregulation of Tcf7 expression during naïve-to-effector differentiation, we identified an effector-associated de novo DNA-methylation program in the Tcf7 locus, suggesting that epigenetic silencing of this gene occurs during the effector stage of the immune response. Furthermore, loci-specific methylation analysis of the DMR in the Tcf7 locus in virus-specific CD8 T cells isolated from chronically infected WT and cKO mice revealed that exhausted WT cells retained a repressive program acquired during the effector stage. Together, these data further support the notion that de novo DNA-methylation programs reinforce commitment to terminal differentiation of exhausted T cells.

### Exhaustion-Associated De Novo Methylation Programming Is Coupled to the Limited Proliferative Capacity of Exhausted CD8 T-Cells.

As CD8 T cells are differentiated toward the fully exhausted state, they progressively lose the ability to undergo antigen-dependent and independent proliferation. Preservation of cKO CD8 T-cell quantity during prolonged exposure to high levels of antigen prompted us to assess the epigenetic status of genes associated with the cell's proliferative potential. Antigen-driven proliferation of CD8 T cells is regulated by the transcription factor c-Myc, which is essential for activation-induced metabolic reprogramming and proliferation of naive CD8 T cells. Gene-expression analysis of exhausted T cells has revealed significant downregulation of c-Myc expression. Given the direct impact c-Myc has on cellular proliferation, we further characterized the exhaustion-associated de novo DMR in the Myc locus to determine if it was coupled to the repressed proliferation of exhausted WT CD8 T cells.

Methylation levels of the Myc-DMR were measured in naïve, functional memory and exhausted WT virus-specific CD8 T cells isolated from acutely or chronically infected mice. Loci-specific methylation analysis revealed that the Myc locus undergoes striking demethylation during the effector stage of the immune response, followed by remethylation during chronic antigen exposure. In contrast, memory CD8 T cells generated after acute infection retained their demethylated state.

To determine if methylation status of the Myc locus is coupled to changes in the proliferative potential of the cell, we first asked if the expression of downstream targets of c-Myc were modified in the absence of this de novo program. Using antigen-specific CD8 T cells isolated during the effector or chronic stages of infection, we measured the surface expression of CD98, a downstream metabolic target of c-Myc that acts as a glutamine antiporter to meet the metabolic demands of proliferating CD8 T cells. Coupled to the demethylated state of the Myc locus DMR, the effector antigen-specific CD8 T cells, isolated at 8 dpi, upregulated CD98 expression. Additionally, CD98 expression was retained on both WT and cKO functional memory CD8 T cells obtained from acutely infected mice. In contrast, CD98 expression was downregulated on the exhausted WT cells but was retained at high levels on Dnmt3a-cKO CD8 T cells isolated from chronically infected mice. These data indicate that the de novo methylation programs acquired during T-cell exhaustion negatively impact the expression of downstream targets of c-Myc in the exhausted WT cells.

To further examine whether de novo methylation at the Myc locus is coupled to limited proliferation of the exhausted T cells, we stained virus-specific CD8 T cells for Ki67, a marker of

cell proliferation, at the effector and chronic stages of the immune response. At 8 dpi, both WT and cKO effector CD8 T cells had recently undergone a burst in antigen-driven proliferation and the majority of the antigen-specific CD8 T cells expressed high levels of Ki67.

Parsing the proliferating effector T cells into Tim-3+ and Tim-3−, PD-1+ subsets revealed comparable quantities of proliferating Tim-3+ PD-1+ among WT and cKO cells, whereas the quantity of proliferating Tim-3− PD-1+ cKO effector CD8 T cells was greater than the comparable WT effector subset. We next assessed Ki67 levels among Tim-3+ and Tim-3−, PD-1+ subsets of virus-specific WT and cKO cells at the exhaustion stage of the immune response. The proliferation of virus-specific WT CD8 T cells was substantially reduced after chronic stimulation. Measurement of Ki67 expression among WT and cKO CD8 T cells established that cKO antigen-specific CD8 T cells maintained a significantly higher level of Ki67 in both Tim-3+ and Tim-3- subsets of PD-1+cells.

Furthermore, we observed that the total pool of virus-specific (CD44hi PD-1+) CD8 T-cells in the cKO mice had greater Ki67-expression compared to the total pool of WT virus-specific CD8 T cells. These data suggest that the elevated quantity of cKO cells during persistent infection is coupled to their retained proliferative capacity. The broader implication of these collective data is that Dnmt3a-mediated DNA-methylation programming establishes the major hallmarks of T-cell exhaustion, and is critical to reinforcing the terminal fate commitment of exhausted T cells.

PD-1 Blockade Therapy Does not Erase Exhaustion-Specific DNA-Methylation Programs in Rejuvenated T cells.

Our finding that exhaustion-associated DNA-methylation programs establish a terminal-exhaustion fate prompted us to ask whether PD-1 blockade results in erasure of exhaustion-associated DNA-methylation in rejuvenated T cells. To address this question we treated chronically infected WT mice with anti-PD-L1, isolated the rejuvenated antigen-specific CD8 T cells, and assessed the methylation status of the exhaustion associated DMRs using our newly generated loci-specific assays. As expected, PD-1 blockade treatment significantly increased the quantity of gp33-specific and total polyclonal CD44hi PD-1+ CD8 T cells in WT mice. Quite strikingly, we observed no change in the effector and post-effector de novo DNA-methylation programs despite T-cell expansion. Preservation of the de novo DNA-methylation programs at the IFNγ, Myc, Tcf7, Ccr7, and T-bet loci in the rejuvenated WT CD8 T cells prompted us to more broadly assess the stability of DNA-methylation programming in all Dnmt3a-targeted genes. WGBS was performed on FACS-purified antigen-specific CD8 T cells from the spleens of the PD-1 blockade-treated and untreated WT mice. Only 5964 DMRs between the treated vs untreated WT CD8 T cells were detected among the WT exhausted and WT rejuvenated WGBS data sets. Among the 5964 DMRs only 84 were Dnmt3a-mediated programs, which accounts for less than 2% reprogramming of the total Dnmt3a-mediated exhaustion programs. Specifically, exhaustion-associated DNA-methylation programming across the IFNγ, Myc, Tcf7, and T-bet loci were unchanged in rejuvenated WT CD8 T cells. These data illustrate how remarkably stable the exhaustion-associated de novo DNA methylation programs are.

Given that PD-1 blockade does not erase these programs, and that they play a causal role in restricting effector function, it raises the question of whether these specific epigenetic programs restrict the therapeutic response of PD-1 blockade treatment.

Inhibition of De Novo DNA-Methylation Programming Synergizes with PD-1 Blockade to Enhance Rejuvenation of CD8 T cells.

The fact that the Dnmt3a cKO virus-specific CD8 T cells persist in an environment with viral loads comparable to chronically infected WT mice, as well as remain PD-1+, provided a unique opportunity to test if Dnmt3a-mediated de novo DNA methylation restricts the ability of cells to respond to PD-1 blockade therapy. Chronically infected WT and Dnmt3a cKO mice were treated with anti-PD-L1 for two weeks and the antigen-specific T cell response was measured. Categorically, the cKO virus-specific CD8 T cells experienced striking increases in frequency and quantity during PD-1 blockade compared to WT virus-specific CD8 T cells. The expanded population of cKO T cells was observed not only in the spleen but also in nonlymphoid tissues. Notably, even NP396-specific CD8 T cells, which are normally refractory to PD-1 blockade, underwent a striking expansion in the frequency and quantity after PD-1 blockade treatment in cKO mice. Consistent with our observation that Dnmt3a-deficient CD8 T cells resist terminal differentiation, the expanded virus-specific cKO CD8 T cells retained lower levels of Tim-3 and Eomes and higher level of T-bet after PD-1 blockade treatment. These data unambiguously demonstrate that de novo DNA methylation programs restrict the ability of exhausted T cells to respond to PD-1 blockade therapy.

Because we observed a striking response across multiple LCMV epitope-specific CD8 T-cell populations in cKO mice, we next proceeded to determine if blocking the acquisition of de novo DNA methylation prior to PD-1 blockade therapy preserved TCR repertoire diversity among the responding cells. Chronically infected WT and cKO mice treated with a PD-1 blocking antibody were bled longitudinally and gp33-specific CD8 T cells were single-cell sorted and the TCR (3-chain was sequenced. Based on the sequencing of several hundred individual cells, a Simpson's diversity index of the TCR repertoire was determined. Interestingly, rejuvenated WT CD8 T cells displayed a reduced TCR repertoire diversity among the expanded gp33-specific T cells, suggesting clonal expansion of a limited number of CD8 T cell clones. In contrast, diversity of the GP33-specific TCR repertoire in cKO CD8 T cells was maintained after PD-1 blockade treatment. These data demonstrate that only a small subset of WT CD8 T cells remain responsive to PD-1 blockade while the majority of cKO CD8 T cell clones are responsive to PD-1 blockade treatment.

We next proceeded to determine whether expanded antigen-specific cKO CD8 T cells retained their effector function. Ex vivo stimulation of WT or cKO splenocytes from chronically infected, mock or anti-PD-L1-treated mice was performed and intracellular production of the effector cytokines IFNγ and IL-2 was measured. Indeed the enhanced expansion the cKO CD8 T cells after 2 weeks of PD-1 blockade did not compromise the cell's capacity to produce IFNγ and IL-2. Given that PD-1 blockade in the cKO mice resulted in expansion of an extensive repertoire of LCMV-specific CD8 T cells with retained effector properties, we next proceeded to determine whether the enhanced therapeutic response impacted the viral load. LCMV viral titers from the sera and tissue of chronically infected WT or cKO mice before and after PD-1 blockade therapy were measured. Strikingly, anti-PD-L1 treatment resulted in a significant reduction in the viral burden in cKO mice compared to that in anti-PD-1-treated WT mice (FIG. 6G). This enhanced viral control was observed not only in the blood, but also in lymphoid and nonlymphoid tissues including spleen and liver.

These data collectively provide proof-of-principle that therapeutic approaches designed to erase or block exhaustion-associated DNA methylation programs may synergize with ICB therapy and enhance the therapeutic response.

In Vivo Treatment with a DNA Demethylating Agent Enhances PD-1 Blockade-mediated T-cell Rejuvenation.

We next sought to ask whether therapeutic treatment of chronically infected mice with a DNA demethylating agent prior to PD-1 blockade treatment could enhance PD-1 blockade-mediated expansion of exhausted WT CD8 T cells. To test this we chronically infected WT mice with LCMV and waited for >1 month before starting treatment in order to generate a scenario in which treatment is initiated after the majority of antigen-specific T cells are exhausted. After 1 month of chronic LCMV infection WT mice were administered a low dose of the standard DNA demethylating agent 5-aza-2′-deoxycytidine (decitabine “DAC”; 1.2 mg/kg) every third day for two weeks. Following DAC treatment, the mice were then treated with the PD-L1 blocking antibody. Longitudinal tracking of virus-specific CD8 T cell quantity in the peripheral blood of chronically infected mice showed a striking increase in the quantity of gp33-specific CD8 T cells in mice that received DAC prior to PD-1 blockade therapy compared to those receiving monotherapy. Notably, when we performed the DAC and PD-1 blockade treatments concurrently we did not observe an enhancement in T cell expansion. Further characterization of antigen-specific and total polyclonal virus-specific CD8 T cells in the spleen, lungs, and liver of chronically infected WT mice receiving sequential DAC and PD-1 blockade therapy revealed that the enhanced expansion of virus-specific T cells occurred in both lymphoid and nonlymphoid tissues. These data indicate that therapeutic treatment with a DNA demethylating agent may prime the T cells for greater sensitivity to ICB therapy.

To determine whether the elevated frequencies of virus-specific CD8 T cells after sequential DAC and PD-1 blockade treatment was coupled to enhanced cellular proliferation, we measured Ki67 expression in antigen-specific and polyclonal virus-specific CD8 T cells after sequential DAC/PD-1 blockade treatment of chronically infected mice. Indeed, the increased frequency of virus-specific CD8 T cell after sequential DAC and PD-1 blockade treatment was coupled to a significant increase in Ki67+ virus-specific CD8 T cells. Collectively, these data indicate that de novo DNA methylation programming in exhausted T cells restricts the efficacy of PD-1 blockade therapy, and strategies to reverse or inhibit these programs may enhance the ICB-responsive potential of T cells.

### Mice and Viral Infections

WT C57BL/6 mice were purchased from Jackson Laboratory. Dnmt3a cKO mice were generated by crossing floxed Dnmt3a mice with mice expressing a Granzyme b-driven recombinase transgene. For chronic infection, WT and cKO mice were treated with 0.75 mg GK1.5 antibody (Harlan Bioproducts) 2 days prior to and on the day of infection to deplete CD4 T cells (Ha et al., 2008a). CD4-deficient mice were then infected with the LCMV clone 13 (2×106 pfu, i.v.). Serum and tissue virus titers were determined by plaque assay. For acute infection, mice were infected with the Armstrong strain of LCMV (2×105 pfu, i.p.).

### In Vivo Decitabine (DAC) and PD-1 Blockade Treatment

Mice were chronically infected with LCMV clone 13 after CD4 T cell depletion as described above. At ≥33 dpi mice were i.p. injected with vehicle or decitabine (Sigma-Aldrich; 1.2 mg/kg) dissolved in sterile PBS every 3 days for 2 weeks. After 2 week of vehicle or DAC treatment, mice were then treated with PBS or anti-PD-L1 (BioXcell; 200 μg) every 3 days for 2 weeks.

### Analysis of Antigen-Specific T-Cell Function and Phenotype

Antigen-specific CD8 T cells were identified or purified by FACS using fluorescently labeled H-2Db tetramers bound to LCMV peptides. LCMV monomers were obtained from the Yerkes NIH tetramer core facility. Phenotypic analysis of the cells was performed using the following fluorescently labeled antibodies: CD8, CD44, CD62L, PD-1, Tim-3, and CD98 (BioLegend). Ki67, T-bet, and Eomes intracellular staining was performed using the eBioscience ICS-staining protocol and fluorescently labeled antibodies against T-bet (clone 4B10; BioLegend), Eomes (clone Dan11mag; eBioscience), and Ki67 (clone SolA15; eBioscience). Ex vivo stimulation of antigen-specific splenocytes was performed using gp33 peptide, as previously described (Youngblood et al., 2011). Intracellular staining for IFNγ, IL-2, and Granzyme b was performed with fluorescently labeled antibody clones XMG1.2, JES6-5h4, and GB11, respectively (all from BioLegend) and by using BD Cytofix/Cytoperm™ (BD Biosciences) per the manufacturer's instructions. Cell death frequency was calculated using Ghost Dye Violet 510 viability dye (Tonbo Biosciences) to determine the frequency of live cells in the total lymphocyte-singlet gate. Data were analyzed using Prism 6 software. Statistical significance was determined using the two-tailed unpaired Mann-Whitney test to compare two to three experiments that used three or more mice per group.

### Genome-Wide and Loci-Specific Methylation Analysis

We sorted viable naïve (CD44low CD62L+) and tetramer+ (gp33+ CD44hi) CD8 T cells from the splenocytes of acutely or chronically infected WT and cKO mice. DNA was isolated from the FACS-purified naïve and antigen-specific CD8 T cells by using the Qiagen DNeasy kit. Genomic DNA was bisulfite treated using the EZ DNA methylation kit (Zymo Research). Bisulfite-induced deamination of cytosine allows for sequencing-based discrimination of methylated vs non-methylated cytosine. To perform genome-wide methylation analysis, a bisulfite-modified DNA-sequencing library was generated using the EpiGnome™ kit (Epicentre) per the manufacturer's instructions. Bisulfite-modified DNA libraries were then sequenced usingan 11lumina Hiseq system. Sequencing data were aligned to the mm10 mouse genome using BSMAP. CpG methylation was performed using model-based analysis of bisulfite sequencing. Differentially methylated regions (DMRs) were identified using Bioconductor package DSS and custom R scripts. The M-value, the measurement of CpG methylation status, was used for PCA and dendrogram. The top 3000 most variable CpG sites were selected to do the principle component analysis (PCA) and clustering analysis.

To perform loci-specific methylation analysis, the bisulfite-modified DNA was PCR amplified with locus-specific primers. The PCR amplicon was cloned into the pGEM-T TA cloning vector (Promega) and then transformed into XL10-Gold ultracompetent E. coli bacteria (Stratagene). Individual bacterial colonies were grown overnight over Luria-Bertani (LB) agar containing ampicillin (100 mg/L), X-gal (80 mg/L), and IPTG (20 mM). White colonies were selected and subcultured into LB broth with ampicillin (100 mg/L) overnight; the cloning vector was purified; and the genomic insert was sequenced.

### TCR Repertoire and Simpson's Diversity Index Analysis

LCMV gp33-specific T cells were stained with specific tetramer, resuspended in freshly made sort buffer (PBS containing 0.1% BSA (Gibco) and 200 U RNAsin/ml (Promega)) and filtered prior to sorting. Tetramer-positive T cells were single cell sorted into the wells of a 96-well PCR plate (Eppendorf) that had been preloaded with 2.5 ul of reverse transcription mixture (0.5 μ1 5X iScript reaction mix, 0.5 μl iScript reverse transcriptase (Biorad), and 0.1% Triton X-100 (Sigma-Aldrich)) using a iCyt Synergy cell sorter (Sony). The parameters used for sorting are: Multi-drop sort OFF, Multi-drop exclude OFF, Division 10, Center sort %: 90. The last two columns of the plate were left unsorted to serve as negative controls for the PCR. Following sorting, the plates were sealed immediately using plate sealer film (MicroAmp, Applied Biosystems) and centrifuged at 500 g for 3 minutes prior to storing at −80° C. until reverse transcription and PCR.

The CDR313 region of individual cells were amplified and sequenced using a nested, single-cell, multiplex PCR approach (Dash et al., 2011). Briefly, cDNA was synthesized directly from single cells as per the manufacturer's instructions with minor modifications. The cDNA synthesis was followed by two rounds of PCR with a Taq polymerase-based PCR kit (Qiagen) and a cocktail of TCR 13 specific primers to amplify the CDR313 transcripts from each cell in a 25-μ1 reaction volume. The PCR products were visualized on a 2% agarose gel, then purified using exonuclease/Shrimp alkaline phosphatase enzymes (Dash et al., 2015) and sequenced using TRBC reverse primer, using an ABI Big Dye sequencer (Applied Biosystem) at the Hartwell Center of St. Jude Children's Research Hospital. The sequence data were analyzed using a custom-built macro-enabled excel sheet in conjunction with an IMGT web interface to derive CDR3β nucleotide and amino acid sequences with corresponding TRBV-TRBJ family usage. Simpson's Diversity Index (D) was calculated on the population from each mouse as previously described (Thomas et al., 2013), with D=Σi[(ni(ni−1))/(N(N−1))], where ni is the number of sequences in the ith clonotype and N is the total number of sequences in the whole population.

### Example 2

### Human Memory CD8 T-cell Effector-potential is Epigenetically Preserved During In Vivo Homeostasis.

Immunological memory is a cardinal feature of adaptive immunity that provides a significant survival advantage by protecting individuals from previously encountered pathogens. Memory CD8 T cells, in particular, have the potential to provide life-long protection against pathogens containing their cognate epitope and are currently being exploited for strategies to protect against various intracellular pathogens and cancer cells. To achieve such long-lived protection, an adequate number of functionally competent memory CD8 T cells must be sustained in the absence of antigen through cytokine-driven homeostatic proliferation. Homeostasis of memory CD8 T cells is predominantly mediated by IL-7 and IL-15-induced expression of pro-survival genes and cell cycle regulators respectively. However, the cell-intrinsic mechanism(s) underlying stable maintenance of acquired effector functions during homeostatic proliferation remains largely unknown. Mounting evidence suggests that DNA-methylation programming is a primary mediator for preserving transcriptionally repressive and permissive chromatin states in cells that have undergone several rounds of division. Therefore, to gain insight into the potential epigenetic basis for maintenance of acquired properties among human memory CD8 T cells whole-genome bisulfite sequencing (WGBS) of sorted primary human naïve, shorter-lived Tem, and long-lived Tcm and Tscm CD8 T cells from healthy donors was performed.

Our initial assessment of genome-wide DNA methylation levels revealed that the overall number of methylated CpGs was inversely correlated with the established differentiation state of these cells: naïve>Tscm>Tcm>Tem. Moreover, the progressive decline in DNA methylation occurred across all autosomal chromosomes, indicating that effector and memory T cell differentiation is coupled to broad changes in DNA methylation. The higher level of methylation among long-lived memory CD8 T cells prompted us to further assess the relationship between naïve and memory CD8 T cell methylation profiles. An unsupervised principal component analysis (PCA) was performed on the methylation status of all CpG sites across the genome. Clustering was also observed among the naïve replicates as well as among T_{scm} replicates; importantly, the naïve and T_{scm} samples were found to be epigenetically distant. On the basis of the methylation status at 9,377,480 CpGs (CpG sites with >5× sequencing coverage for every sample), we generated a dendrogram of all replicate samples. Calculation of Euclidean distances between each population in the dendrogram indicated that despite the higher level of global DNA methylation, long-lived memory CD8 T cells (Tscm) have DNA methylation programs that are distinct from naïve cells.

To better define the DNA methylation programs that distinguish memory CD8 T cells from naïve cells we performed a pair-wise comparison of naïve versus memory cell WGBS datasets identifying differences in DNA methylation at individual CpG sites across the genome. This comparison allowed us to define the number, distribution, and nature of differentially methylated regions (DMRs) between the genomes of naïve and memory T cell subsets. We observed the greatest number of demethylated regions in Tₑₘ cells relative to naïve T cells, followed by Tₑₘ cells, and then T_{scm} cells, further confirming our PCA results that the Tₑₘ memory subset are the most epigenetically distinct population from naïve CD8 T. Regardless of the methylated versus demethylated status, the majority of the DMRs were enriched in the 5′-distal regions (1-50 Kb) suggesting an association with transcriptional regulatory regions.

We next sought to identify DNA methylation programs coupled to the unique properties of the individual memory T cell subsets. Again a pair-wise comparison of the methylation status between each memory subset was performed and we detected 201980, 62240, and 9026 DMRs unique to Tₑₘ, T_{cm}, and T_{scm} CD8 T cells respectively. Among the DMRs that delineate the Tₑₘ, T_{cm}, and T_{scm} CD8 T cells were subset-associated DMRs at CpG sites in the CCR7 and CD62L (SELL) loci. Both CCR7 and CD62L DMRs were significantly methylated in CD8 Tₑₘ, cells while these regions remained predominantly unmethylated in naïve, Tₑₘ, and T_{scm} CD8 T cells, consistent with the relative level of expression of these molecules in the different cell subsets. Similar to the lymphoid-homing molecules, we observed striking differences in methylation status at the transcription factor loci for T-bet and eomesodermin (Eomes), both of which have well-established roles in CD8 T-cell effector and memory differentiation. Consistent with the relative level of gene expression, all memory CD8 T cells were generally demethylated at regions downstream of the TSS of T-bet and Eomes, relative to that in naïve T cells. Notably, the Eomes locus contained a greater level of methylation in T_{scm} cells relative to the Tₑₘ, cells at each of the DMRs.

In contrast to the memory subset-specific DNA methylation programs found at lymphoid homing molecules and transcription factors, demethylation DMRs at loci of classically defined effector molecules including IFN□, Perform, GzmB, and GzmK were observed in all memory T cell subsets compared to naïve cells. Of particular note was the striking level of demethylation at these loci in the long-lived T_{scm} CD8 T cells. To more broadly characterize DMRs linked to memory T cell longevity, we performed an ingenuity pathway analysis (IPA) of gene associated with T_{scm} DMRs. The IPA upstream regulator analysis identified STAT3 among the top potential regulators of the T_{scm} DMR gene list, further linking memory CD8 T cell development and the epigenetic poising of effector function in long-lived memory T cells.

Having determined that the loci of several effector molecules in long-lived memory CD8 T cells contain an epigenetic program suggestive of transcriptional permissivity, we next sought to determine if the effector-associated loci were poised for rapid gene expression in response to TCR stimulation. Naïve and memory CD8 T-cell subsets were purified and then cultured in the presence of anti-CD3/CD28 antibodies. mRNA was isolated longitudinally from the naïve and memory CD8 T cell subsets at 0, 4, and 12 hours following stimulation and the level of IFNγ, GzmB, and Prf1 transcription after TCR stimulation was determined. Our results revealed that GZMB and PRF1 transcription is rapidly induced in T_{cm}, and T_{scm} cells upon TCR ligation, while Tₑₘ cells maintained a constitutively high level of expression following TCR activation. Interestingly, the level of IFNγ mRNA was high in all resting memory CD8 T cell subsets relative to naïve cells but was further upregulated upon stimulation of the memory subsets. Similar to the heightened kinetics for gene expression, TCR stimulation of the purified memory CD8 T-cell subsets also resulted in a rapid increase in the production of GzmB in T_{cm}, and T_{scm} cells, relative to that in naïve T cells. These results provide further evidence that the epigenetic status for the IFNγ, PRF1, and GZMB genes in T_{cm} and T_{scm} cells is coupled to the poising of effector molecule expression.

To further assess the ability of memory CD8 T-cell subsets to maintain a “poised-for-expression” gene expression program during antigen-independent proliferation, we measured the expression of IFNγ following in an in vitro model of homeostatic cytokine-driven cell proliferation. Purified naïve and memory CD8 T cell subsets were labeled with the cell proliferation tracking dye CFSE, and then cultured in the presence of the homeostatic cytokines IL-7 and IL-15 for 7 days. Indeed, our results confirm prior reports of human memory CD8⁺ T-cell subsets having a hierarchical capacity to undergo cytokine driven homeostatic proliferation, with T_{scm} cells having the highest level of proliferation to both cytokines (naive<Tₑₘ<T_{cm}<T_{scm}, having undergone three or more cell divisions). We next measured the poised-recall response in cells that had undergone cytokine-driven proliferation by assessing the level of IFNγ protein in undivided and divided CD8 T cells after TCR stimulation. Quite strikingly, after 7 days in culture with IL-7 and IL-15, divided memory CD8 T cells retained the ability to express elevated levels of IFNγ protein after 4 hours TCR stimulation. The results suggest that human memory CD8 T cells retain a gene expression program during IL-7/IL-15 mediated proliferation that allows the cells to remain poised to elicit a rapid effector response.

Our WGBS methylation analyses of primary T cells serves as a “snapshot” of the epigenetic state of long-lived memory CD8 T cells but fails to reveal whether or not the DNA-methylation programs are stable during homeostasis. Having validated that DNA methylation status of many of the DMRs identified from our WGBS analyses, including the DMRs identified in the IFNγ and Prf1 loci, we proceeded to use our newly designed loci-specific assays to determine whether the methylation status would remain unchanged during in vitro cytokine-driven homeostatic proliferation. Naïve, Tₑₘ, T_{cm}, and T_{scm} CD8 T cell subsets were FACS purified, labeled with CFSE, and then maintained in culture with IL-7 and IL-15 for 7 days. After 7 days, we then FACS purified the undivided and divided (>3 rounds of cell division) fraction of cells and measured their DNA-methylation status. The IFNγ locus remained fully demethylated in all memory T-cell subsets that had undergone cell division, compared to naïve CD8 T cells. Moreover, naïve CD8 T cells that underwent more than three rounds of division retained a fully methylated IFNγ locus. These data demonstrate that cell division alone is not sufficient to demethylate the IFNγ locus in naïve cells; rather the process of demethylation is coupled to additional events/stages of memory T-cell differentiation.

Similar to the IFN□ locus, the demethylated status of CpGs within the Prf1 locus remained unchanged in dividing CD8 Tₑₘ cells. This region of the Prf1 locus was approximately 50% demethylated in resting CD8 T_{cm} and T_{scm} cells, which enabled us to test whether memory T cells undergo further demethylation through passive mechanisms (i.e., failure to propagate a methylation program during cell division). Remarkably, the 50% methylation status at the CpG sites in the T_{cm} and T_{scm} cells was faithfully propagated for more than three rounds of cell division, demonstrating that acquired epigenetic programs at effector-associated loci can persist during cytokine-drive homeostatic proliferation.

Antigen-independent phenotypic conversion of memory CD8 T cells occurs during in vivo and in vitro homeostatic proliferation but it remains openly debated whether this phenotypic conversion represents bone fide reprogramming of the cell's differentiation state. Indeed, culturing naïve, Tₑₘ, T_{cm}, and T_{scm} CD8 T cells with IL-7/IL-15 for 7 days results in a down-regulation of CCR7 expression in both T_{cm} and T_{scm} and a conversion to Tₑₘ-like cells. This observation promoted us to investigate the status of DNA methylation in CCR7 and CD62L DMRs under these conditions. We first confirmed that the CpG sites in the CCR7 and CD62L DMRs were fully demethylated in both naïve and T_{scm} cells and significantly methylated in Tₑₘ cells isolated from six independently sorted samples. These data further substantiate the link between CCR7 and CD62L expression and the methylation status of the DMRs. We next measured the methylation status of CCR7 and CD62L CpGs during cytokine-driven proliferation using the loci-specific assay. Naïve and memory CD8 T cell subsets were again cultured in the presence of IL-7 and IL-15 and the methylation assay was performed on purified undivided and divided populations. Similar to our findings with the IFNγ and Prf1 DMRs, the methylation status of the CCR7 and CD62L DMR CpGs in divided naïve CD8 T cells remained unchanged. However, we detected a significant increase in the methylation levels at the CCR7 DMR in divided T_{scm} cells. These results provide compelling evidence that cytokine-induced developmental changes among long-lived memory CD8 T cells are coupled to the cell's ability to undergo selective epigenetic reprogramming.

Collectively, the results from our in vitro homeostasis studies establish that DNA methylation programs associated with the heightened recall of effector functions are preserved over several rounds of cytokine-driven cell division, while programs coupled to homing and broadly used to delineate memory T cell subsets, can be modified. Although the effector-associated epigenetic programs exhibited remarkable stability under conditions of in vitro homeostasis, a lingering question is whether such stability occurs in vivo. One of the main challenges of studying in vivo human T cell homeostasis is the difficulty of tracking and re-isolating adoptively transferred T cells from the recipient due to their low frequency in circulation and the lack of congenic markers to distinguish donor versus recipient T cells. To overcome these challenges we took advantage of a novel T-cell depletion strategy utilized at our institution that selectively depletes CD45RA+ cells in haploidentical donor grafts for hematopoietic cell transplantation, thereby providing adoptive transfer of numerous donor memory cells at the time of transplantation. This infusion of polyclonal total Tcm and Tem memory T cells provides a unique opportunity to assess stability of epigenetic programs in human memory CD8 T cells during in vivo homeostatic proliferation.

Using the transplantation procedure we proceeded to assess the stability of DNA methylation programs in memory CD8 T cells that underwent antigen-independent expansion in vivo. Five blood samples from hematopoietic cell transplant recipients were selected for analyses based on the criteria of 100% donor chimerism among the reconstituted immune cells after infusion and no signs of immunological responses to infection. Donor T cells were phenotypically characterized prior to CD45RO enrichment for adoptive transfer and then characterized again ˜2 months after adoptive transfer and expansion in the patient. CD8 T cells isolated from the blood of recipients were strikingly void of cells exhibiting a naïve phenotype indicating that enrichment prior to infusion indeed excluded CD45RO— cells. The expanded CD8 T cells predominantly exhibited a Tₑₘ phenotype, despite the transfer of both T_{cm}, and Tₑₘ memory CD8 T cell, and also expressed significantly higher levels of Ki67 indicating that they had recently proliferated. Notably, memory CD8 T cells isolated from the recipients had only a modest increase in the level of PD-1 expression, further supporting the conclusion that the majority of memory T cells in these patients had not recently encountered pathogen-associated antigens.

Having established that the majority of T cells isolated from the PBMCs of recipients retained a memory phenotype and originated from the donor (chimerism was 100% based on VNTR), we next sought to determine the DNA methylation status of effector and homing-associated DMRs in these cells. Loci-specific DNA methylation profiling of the IFNγ and Prf1 DMRs in purified donor Tem CD8 T cells (pre-transfer) and Tem-phenotyped cells isolated from the recipients confirmed that the promoters of these effector-associated genes remained demethylated during in vivo memory T cell reconstitution of the recipients. These data unambiguously establish that memory T cells can maintain a transcriptionally permissive epigenetic program at effector-associated loci during in vivo antigen-independent proliferation. Additionally, the CCR7 and CD62L DMRs were heavily methylated in the recipient memory T cells compared to the input donor memory T cells. Therefore, despite the donor infusion containing both T_{cm}, and Tₑₘ, CD8 T cells, the recipient was found to have primarily Tₑₘ CD8 T cells. It is quite possible that the absence of Tcm-like CD8 T cells from the circulation of the recipients' samples was due to selective death of the transferred T_{cm}, or selective homing to the lymphoid tissue. Yet, a more exciting possibility is that these data represent in vivo evidence of memory CD8 T cell subset inter-conversion. Such conversion of T_{cm}, CD8 T cells into cells with a Tem phenotype is consistent with our in vitro results showing that gamma chain cytokines promote the conversion of long-lived memory CD8 T cells into Tem memory CD8 T cells.

Over the lifetime of an organism, memory T cell homeostasis ensures protection against pathogens that the host was previously exposed to and is achieved in part, by a fine balance between the death and proliferation of those cells. This balance is largely orchestrated by the common cytokines IL-7, which is essential for cell survival, and IL-15, which promotes cell cycling. Our study establishes that in vivo preservation of effector potential during cytokine-mediated homeostasis of memory CD8 T cells is coupled to the ability of the cell to transcribe acquired DNA methylation programs to newly generated daughter cells. Moreover, these results reveal that stabilization of epigenetic programming occurs in a loci-specific manner, providing new insight into the mechanisms regulating memory T cell subset inter-conversion. Broadly these data highlight epigenetic programming as a mechanism memory T cells use to strike a balance between remaining adaptive to their current and future environment while also retaining a history of past events.

Isolation of human CD8 T cells from healthy donor blood: This study was conducted with approval from the Institutional Review Board of St. Jude Children's Research Hospital. Human peripheral blood mononuclear cells (PBMCs) were collected through the St. Jude Blood Bank, and samples for WGBS were collected under IRB protocol XPD15-086. PBMCs were purified from platelet apheresis blood unit by density gradient. Briefly, blood was diluted 1:2.5 using sterile Dulbecco's phosphate-buffered saline (Life Technologies). The diluted blood was then overlayed above Ficol-Paque PLUS (GE Healthcare) at a final dilution of 1:2.5 (ficoll:diluted blood). The gradient was centrifuged at 400×g with no brake for 20 minutes at room temperature. The PBMCs interphase layer was collected and washed with 2% fetal bovine serum (FBS)/1 mM EDTA PBS buffer and then centrifuged at 400×g for 5 minutes. Total CD8 T cells were enriched from PBMCs by using the EasySep™ human CD8 negative selection kit (EasySep™, STEMCELL Technologies). Donors and patients were enrolled on an IRB approved protocol (registered at ClinicalTrials.gov, Identifier: NCT01807611), and provided informed consent for collection of the blood samples used for the in vivo analyses. Donor chimerism was determined utilizing CLIA-certified VNTR analysis.

Isolation and flow cytometric analysis naïve and memory CD8 T-cell subsets: Following enrichment of CD8 T cells, naïve and memory CD8 T-cell subsets were sorted using the following markers as previously described (23, 31). Naïve CD8 T cells were phenotyped as live CD8⁺, CCR7⁺, CD45RO⁻, CD45RA⁺, CD95⁻ cells. CD8 Tₑₘ cells were phenotyped as live CD8⁺, CCRT, CD45RO⁺ cells. T_{cm} cells were phenotyped as live, CD8⁺, CCR7⁺, CD45RO⁺ cells. T_{scm} cells were phenotyped as live CD8⁺, CCR7⁺, CD45RO⁻, CD95⁺ cells. Sorted cells were checked for purity (i.e., samples were considered pure if more than 90% of the cells had the desired phenotype). Granzyme B expression was measured using sorted naïve or memory CD8 T-cell subsets stimulated with Dynabeads human T-cell activator CD3/CD28 at a 1:1 ratio. After approximately 18 hours of incubation at 37° C. and 5% CO₂, cells were harvested for cell-surface staining followed by intracellular staining.

Genomic Methylation Analysis: DNA was extracted from the sorted cells by using a DNA-extraction kit (Qiagen) and then bisulfite treated using an EZ DNA methylation kit (Zymo Research), which converts all unmethylated cytosines to uracils, while protecting methylated cytosines from the deamination reaction. The bisulfite-modified DNA-sequencing library was generated using the EpiGnome™ kit (Epicentre) per the manufacturer's instructions. Bisulfite-modified DNA libraries were sequenced using an Illumina Hiseq. Sequencing data were aligned to the HG19 genome by using BSMAP software. Differential-methylation analysis of CpG methylation among the datasets was determined using a Bayesian hierarchical model to detect regional methylation differences with at least three CpG sites. To perform loci-specific methylation analysis, bisulfite-modified DNA was PCR amplified with locus-specific primers (Supplemental Table). The PCR amplicon was cloned into a pGEMT easy vector (Promega) and then transformed into XL10-Gold ultracompetent bacteria (Stratagene). Bacterial colonies were selected using a blue/white X-gal-selection system after overnight growth, and then the cloning vector was purified and the genomic insert was sequenced. Following bisulfite treatment, the methylated CpGs were detected as cytosines in the sequence, and unmethylated CpGs were detected as thymines in the sequence by using BISMA software.

In vitro homeostatic proliferation: Sorted naïve CD8 T cells or memory CD8 T-cell subsets were labeled with CFSE (Life Technologies) at a final concentration of 2 μM. CFSE-labeled cells were maintained in culture in RPMI containing 10% FBS, penicillin-streptomycin, and gentamycin. Cells were maintained in culture with IL-7/IL-15 at a final concentration of 25 ng/mL each. After 7 days of incubation at 37° C. and 5% CO₂, undivided and divided cells (third division and higher) were sorted. Sorted cells were checked for purity (>90%). To determine whether the effector-recall response was maintained, we stimulated naïve and memory CD8 T-cell subsets with anti-CD3/CD28 beads (1:1) ratio for 4.5 hours in the presence of Golgi Stop and Golgi Plug after a 7-day exposure to IL-7/IL-15 in culture and then examined the levels of IFNγ protein expression by intracellular staining. For GzmB, cells were stimulated for 18 hrs with anti-CD3/CD28 beads (1:1) ratio.

Quantitative Transcriptional Analysis: Total RNA was extracted from naïve and memory CD8⁺ T-cell subsets by using RNeasy plus micro kit (Qiagen). RNA was reverse transcribed into cDNA by using Superscript III reverse transcriptase (Roche Applied Science). Real-time PCR was performed on a CFX96 Real-time System (BioRad). Relative quantities of mRNA were determined using the Syber Select Master Mix CFX (Roche Applied Biosciences). Primer sequences are provided in the Supplementary Materials. The levels of mRNA for each gene were normalized to that of β-actin, and the fold increase in signal over naïve CD8 T cells was determined.

1. A method for modulating T-cell activity comprising:
modulating the methylation profile of the genome of a CD8 T cell.

2. The method of claim 1, wherein methylation of the loci of effector cytokines, transcription factors, and regulators of cellular proliferation is altered.

3. The method of claim 1, wherein methylation of the loci of effector cytokines, transcription factors, and regulators of cellular proliferation is decreased.

4. The method of claim 1, wherein said effector cytokines, transcription factors, and regulators of cellular proliferation comprise at least one of: IFNγ, granzyme K, GzmB, and Prf1, T-bet, Tcf7, Myc, T-bet, eomesodermin (Eomes), Foxp1, CCR7, and CD62L.

5. The method of claim 2, wherein the methylation of at least one CpG site within said locus is decreased.

6. The method of claim 5, wherein said at least one CpG site is located within a promoter sequence or transcription factor sequence.

7. The method of claim 6, wherein said promoter sequence or transcription factor sequence is operably linked to a nucleic acid sequence encoding an effector cytokine, transcription factor, or regulator of cellular proliferation.

8. The method of claim 7, wherein said effector cytokine, transcription factor, or regulator of cellular proliferation comprises at least one of: IFNγ, granzyme K, GzmB, and Prf1, T-bet, Tcf7, and Myc.

9. The method of claim 1, wherein modulating the methylation profile comprises contacting said T cell with a demethylation agent to produce a modified CD8 T cell.

10. The method of claim 1, wherein modulating the methylation profile comprises decreasing the activity of at least one DNA methyltransferase to produce a modified CD8 T cell.

11. The method of claim 9, wherein said contacting step occurs in vitro.

12. The method of claim 9, wherein said modified CD8 T cell is administered to a subject.

13. The method of claim 1, wherein said CD8 T cell is a CAR CD8 T cell.

14-16. (canceled)

17. The method of claim 14, further comprising administering an ICB therapy.

18. A method for selecting a subset of CD8 T cells comprising
measuring the methylation profile of at least one CD 8 T cell; and
separating a subset of CD8 T cells comprising at least one positive memory cell methylation marker.

19. The method of claim 18, wherein said positive memory cell methylation marker comprises an unmethylated memory cell methylation marker.

20-22. (canceled)

23. A population of CD8 T cells selected by the method claim 18.

24-29. (canceled)

30. A pharmaceutical composition comprising said population of CD8 T cells of claim 23.

31-37. (canceled)

38. A method of treating a chronic infection or cancer in a subject, said method comprising:
decreasing the activity of at least one DNA methyltransferase in a subject having at least one negative memory cell methylation marker.

39. The method of claim 5, wherein said DNA methyltransferase is Dnmt3a.

40-47. (canceled)
